(19) United States
(12) Patent Application Publication
(10) Pub. No.: US 2016/0108105 A1
(43) Pub. Date: Apr. 21, 2016

(54) HUMAN IGG4 FC POLYPEPTIDE VARIANT

(71) Applicant: Genexine, Inc., Seongnam-si (KR)

(72) Inventors: Se Hwan YANG, Seoul (KR); Eun Ju SHIN, Yongin-si (KR); Jaehan PARK, Seongnam-si (KR); Eun Joo NAM, Seoul (KR)

(73) Assignee: GENEXINE, INC., Seongnam-si (KR)

(21) Appl. No.: 14/875,746

(22) Filed: Oct. 6, 2015

Related U.S. Application Data
(60) Provisional application No. 62/060,200, filed on Oct. 6, 2014.

(51) Int. Cl. C07K 16/00; C07K 14/61

(52) U.S. Cl. CPC C07K 16/00; C07K 14/61; C07K 2317/524; C07K 2317/526; C07K 2317/66; C07K 2319/30; C07K 2317/94

(57) ABSTRACT

Disclosed is a human IgG4 Fc polypeptide variant, including a modified CH2 domain of human IgG4 Fc, wherein the modification contains a replacement of a 6-20 consecutive amino acid sequence from the N-terminus of the CH2 domain of IgG4 with a 4-18 consecutive amino acid sequence from the N-terminus of human IgA1 CH2, a 4-18 consecutive amino acid sequence from the N-terminus of human IgA2 CH2, a 4-18 consecutive amino acid sequence from the N-terminus of human IgD CH2, a 4-18 consecutive amino acid sequence from the N-terminus of human IgE CH2, or a 4-18 consecutive amino acid sequence from the N-terminus of human IgM CH2. The Fc variant imparts to a biologically active polypeptide—the modified Fc variant with a prolonged in-vivo half-life and reduced immunogenic reaction.

### CROSS REFERENCE TO RELATED APPLICATIONS

This application claims the benefit of provisional application No. 62/060,200, filed on Oct. 6, 2014, the content of which is incorporated herein by reference in its entirety.

### BACKGROUND OF THE INVENTION

1. Field of the Invention

The present invention relates to a human IgG4 Fc polypeptide variant, in which the Fc polypeptide is prepared by replacing a portion of the N-terminus of CH2 domain of human IgG4 with a portion of CH2 domain of other immunoglobulin Fc, a chimeric polypeptide comprising the polypeptide and a biologically active molecule, a method for producing the polypeptide and the chimeric polypeptide, a nucleic acid molecule encoding the same, an expression vector comprising the nucleic acid molecule, and a host cell comprising the same.

2. Description of the Related Art

Biologically active molecules may be of great interest therapeutically. However, their circulating half-life or serum half-life is very short because they are digested by various enzymes in living body. Thus, they may have disadvantages as a therapeutic agent. Therefore, many studies have been conducted to improve the circulating half-life of biologically active molecules.

One of the studies is to increase the circulating half-life or to prevent protein degradation by conjugation of polyethylene glycol (PEG) to an active protein or by control of glycosylation of the active protein.

Compared to first-generation proteins, PEGylated proteins have increased half-life by reducing renal clearance or degradation by proteolytic enzymes in the blood, but conjugation of PEG considerably reduces bioactivity of the proteins and additional PEGylation process of the purified proteins is required, leading to an increase in the production cost and a side effect of PEG accumulation in the living body when administered for a long period of time. Glycosylation is a method of increasing half-life due to a remarkable reduction in hepatic clearance by attaching sugars, in particular, sialic acids to specific amino acid sites. However, there are disadvantages that artificial attachment of sialic acids reduces activity and half-life is not greatly increased.

In the 1990s, therefore, chimeric proteins prepared by fusion of immunoglobulin (Ig) as a partner have been studied as a method for increasing in-vivo circulating half-life of biologically active molecules.

Meanwhile, immunoglobulins are composed of four polypeptide chains, two heavy chains and two light chains, which are associated via disulfide bonds to form tetramers.

Each chain is composed of a variable region and a constant region. The constant region of the heavy chain is further divided into three or four regions (CH1, CH2, CH3, and CH4), depending on the isotypes. The Fc portion of the heavy chain constant region, depending on the Ig isotype, includes hinge, CH2, CH3, and/or CH4 domains.

IgG1, IgG2, and IgG4 have long half-lives of 21 days, while other immunoglobulins have half-lives of less than a week. Based on these characteristics of the immunoglobulins, a fusion protein was prepared by fusing Fc portion of IgG having a long half-life to a biologically active protein, and it was confirmed that the prepared immunoglobulin fusion protein shows increased stability and increased serum half-life. Studies regarding this have been actively conducted.

At the beginning period, fusion proteins in which an IgG is coupled or fused to a biologically active substance including s extracellular domains of cell surface receptors such as CD4 (Capon et al., Nature 1989. 327: 525-531), TNFR (Mohler et al., J. Immunology 1993. 151: 1548-1561), CTLA4 (Linsley et al., J Exp. Med. 1991. 173: 721-730), CD 86 (Morton et al., J. Immunology 1996. 156: 1047-1054) have been produced. Also, there are several cytokines and growth hormones which have been fused to Fc or CH domains of IgG.

However, unlike the fusion with the extracellular domains of cell surface receptors, a fusion with soluble proteins to IgGs leads to reduced biological activities, compared to the non-fused cytokine or growth factors.

The immunoglobulin fusion proteins exist as dimers, which lead to the steric hindrance from the interacting with their target molecules like receptors, due to the presence of two active proteins in close proximity to one another. Therefore, this problem should be overcome to make an efficient Fc fusion protein.

The other limitation of the Fc fusion technology is the presence of undesirable immune responses. The Fc domain of the immunoglobulin has also effector functions such as antibody dependent cell-mediated cytotoxicity (ADCC) or complement-dependent cytotoxicity (CDC). This effector functions are generally achieved via interaction between the Fc region of the Ig and FcγR on effector cells or via complement binding. Therefore, the blocking of effector functions of Fc should be performed to reduce the undesirable reactions such as cell killing, cytokine release, or inflammation.

Many trials have been conducted to prepare Fc fusion proteins using the immunoglobulin IgG1, IgG2 or IgG4 having a long in-vivo half-life, compared to other immunoglobulins. However, these proteins also induce undesirable immune reactions due to effector functions such as ADCC or CDC.

To overcome these limitations, it has been tried that the ADCC or CDC-inducing region of the immunoglobulin is artificially modified, and fused to biologically active proteins, thereby prolonging the half-life of the biologically active protein. However, the artificial mutations within the immunoglobulins may also induce undesirable immune responses, and thus the fused proteins are not suitable for a long-term treatment.

Accordingly, there is still needs for an improved IgG or IgG Fc variant which can be fused to a biologically active peptide to give a fused peptide which has an increased half-life without or with a reduced immunogenic reaction in a subject.

The present inventors provide an IgG4 Fc variant which comprises a modified IgG4 CH2 domain and an IgG4 CH3 domain, wherein the modification of the IgG4 CH2 domain comprises a replacement of a portion of the N-terminus of IgG4 CH2 domain by an N-terminus portion of IgA, IgD, IgE, or IgM. which induces ADCC. In this regard, a native form of the immunoglobulin having no mutation was used as it is, and the junction site in the substituted region was designed to have a hydrophobic property so that the junction site formed in vivo is not exposed to the exterior, thereby minimizing undesirable non-specific immune responses.

Consequently, an object of the present invention is to develop a CH2 domain variant of IgG4 Fc, which is able to increase half-life of a physiologically active protein and to minimize non-specific immune responses without inducing ADCC.

### SUMMARY OF THE INVENTION

An object of the present invention is to provide a human IgG4 Fc polypeptide variant, in which the Fc polypeptide is prepared by replacing a portion of CH2 domain of human IgG4 with a portion of CH2 domain of immunoglobulin Fc of other class.

Another object of the present invention is to provide a chimeric polypeptide comprising the polypeptide and a biologically active molecule.

Still another object of the present invention is to provide a method for producing the polypeptide or the chimeric polypeptide.

Still another object of the present invention is to provide a nucleic acid molecule encoding the polypeptide or the chimeric polypeptide.

Still another object of the present invention is to provide an expression vector comprising the nucleic acid molecule.

Still another object of the present invention is to provide a host cell comprising the expression vector.

### BRIEF DESCRIPTION OF THE DRAWINGS

FIG. 1 shows a comparison of in-vivo half-life between hGH fused with the wild-type IgG4 Fc (hGH-IgG4Fc-wt) and a control group somatropin;

FIG. 2 shows a comparison of FcγRI-binding ability between hGH fused with the wild-type IgG4 Fc (hGH-IgG4Fc-wt) and a control group rituxan;

FIG. 3 shows a process of searching a portion to be removed from the N-terminus of IgG4 Fc CH2 domain;

FIG. 4 shows the results of sequence alignments of CH2 domain of IgG4 and CH2 domains of other immunoglobulins (IgG1, IgG3, IgG2, IgE, IgA1, IgA2, IgM and IgD);

FIG. 5 shows FcγRI-binding ability of an IgG4 variant which was prepared by replacing a portion of the N-terminus of IgG4 Fc CH2 domain with a portion of IgD CH2 domain;

FIG. 6 shows in-vivo half-life of the IgG4 variant which was prepared by replacing a portion of the N-terminus of IgG4 Fc CH2 domain with a portion of IgD CH2 domain;

FIG. 7 shows diagrams of different IgG4 Fc CH2 variants which were prepared by replacing a portion of the N-terminus of IgG4 Fc CH2 domain with a portion of IgA1, IgA2, IgD, IgE, IgM CH2 domain;

FIG. 8 illustrates different IgG4 Fc CH2 variants which were prepared by replacing a portion of the N-terminus of IgG4 Fc CH2 domain with a portion of IgA1, IgA2, IgD, IgE, IgM CH2 domain;

FIG. 9 shows diagrams of different IgG4 Fc CH2 variants which were prepared by replacing a portion of the N-terminus of IgG4 CH2 domain with a portion of IgA1, IgA2, IgE, IgM CH2 domain;

FIG. 10 illustrates different IgG4 Fc CH2 variants which were prepared by replacing a portion of the N-terminus of IgG4 CH2 domain with a portion of IgA1, IgA2, IgE, IgM CH2 domain;

FIG. 11 shows in-vivo half-life of each of the different IgG4 Fc CH2 variants which were prepared by replacing a portion of the N-terminus of IgG4 CH2 domain with a portion of IgA1, IgA2, IgE, IgM CH2 domain;

FIG. 12 shows FcγRI-binding ability of each of the different IgG4 Fc CH2 variants which were prepared by replacing a portion of the N-terminus of IgG4 CH2 domain with a portion of IgA1, IgA2, IgE, IgM CH2 domain;

FIGS. 13a to 13e show a hydrophobicity profile of each of the different IgG4 Fc CH2 variants which were prepared by replacing 10 amino acid residues of the N-terminus of IgG4 CH2 domain with 8 amino acid residues of IgA1 (FIG. 13a), IgA2 (FIG. 13b), IgD (FIG. 13c), IgE (FIG. 13d), or IgM (FIG. 13e) CH2 domain; and

FIGS. 14a, 14b, 15a, 15b, 16a, 16b, 17a, 17b, 18a, and 18b show a hydrophobicity profile of each of the different IgG4 Fc CH2 variants which were prepared by replacing 20 or 21 amino acid residues of the N-terminus of IgG4 CH2 domain with 18 or 19 amino acid residues of IgA1 (FIGS. 14a and 14b), IgA2 (FIGS. 15a and 15b), IgD (FIGS. 16a and 16b), IgE (FIGS. 17a and 17b), or IgM (FIGS. 18a and 18b) CH2 domain.

### DETAILED DESCRIPTION OF THE PREFERRED EMBODIMENTS

In one aspect to achieve the above objects, the present invention provides a human IgG4 Fc polypeptide variant, in which a portion of CH2 domain of human IgG4 is replaced with a portion of CH2 domain of immunoglobulin Fc of other class.

In detail, the present invention provides a human IgG4 Fc polypeptide comprising a hinge region, a modified CH2 domain of human IgG4 Fc, and a CH3 domain of human IgG4 Fc in an N-terminal to C-terminal direction, in which the modification to the CH2 domain comprises a replacement of a portion of the N-terminus of CH2 domain of IgG4 with a portion of CH2 domain selected from the group consisting of CH2 domain of human IgA1 Fc, CH2 domain of human IgA2 Fc, CH2 domain of human IgD Fc, CH2 domain of human IgE Fc and CH2 domain of human IgM Fc. The polypeptide is prepared by replacing a portion of CH2 domain in the wild-type (native form) human IgG4 Fc polypeptide, and referred to as, herein, human IgG4 Fc polypeptide variant, human IgG4 Fc mutant polypeptide, modified human IgG4 Fc region, or human IgG4 Fc region variant. In addition, it can be used interchangeably with Fc polypeptide or Fc region herein.

In the present invention, the human IgG4 Fc polypeptide has an advantage of increasing half-life of a biologically active molecule by binding to the biologically active molecule, but a disadvantage that the portion of CH2 domain of IgG4 Fc induces an undesirable non-specific immune response, ADCC (Antibody-dependent cellular cytotoxicity). Therefore, the present inventors replaced the portion of the CH2 domain of IgG4 Fc with a portion of CH2 domain of immunoglobulin Fc of other class so as to effectively inhibit an ADCC-inducing ability which becomes a disadvantage as a fusion partner of the biologically active molecule, while maintaining the advantage of a long half-life of IgG4 Fc domain of the native form. Further, because the IgG4 Fc domain is not artificially mutated or not replaced with any sequence, but is replaced with a portion of Fc domain of other different class belonging to human immunoglobulin, there is no safety problem in the human body and non-specific immune response is minimized, thereby maintaining the therapeutic effect of the biologically active protein for a long period of time without side-effect such as cytotoxicity, etc.

As used herein, the term “Fc fragment,” “Fc region,” or “Fc” refers to a peptide that includes the heavy-chain constant region (CH) of an immunoglobulin, and does not include the variable regions of the heavy and light chains, and the light-chain constant region (CL) of the immunoglobulin. The Fc may further include the hinge region, and with respect to the objects of the present invention, it may include the heavy chain constant region 2 (CH2) and the heavy chain constant region 3 (CH3), but may include or may not include the heavy chain constant region (CH1).

In addition, the Fc fragment of the present invention may be in the form of having native sugar chains, increased sugar chains compared to a native form or decreased sugar chains compared to the native form, or may be in a deglycosylated form. The increase, decrease or removal of the immunoglobulin Fc sugar chains may be achieved by methods common in the art, such as a chemical method, an enzymatic method and a genetic engineering method using a microorganism. The removal of sugar chains from an Fc fragment results in a sharp decrease in binding affinity to the C1q part of the first complement component C1 and a decrease or loss in antibody-dependent cell-mediated cytotoxicity (ADCC) or complement-dependent cytotoxicity (CDC), thereby not inducing unnecessary immune responses in vivo. In this regard, an immunoglobulin Fc fragment in a deglycosylated or aglycosylated form may be, in some cases, more suitable to the object of the present invention as a drug carrier.

As used herein, the term “deglycosylation” means that sugar moieties are enzymatically removed from an Fc fragment, and the term “aglycosylation” means that an Fc fragment is produced in an unglycosylated form by a prokaryote, preferably E. coli.

In the present invention, the amino acid sequences of the human immunoglobulin and Fc thereof are well known in the art and they are deposited with a publicly accessible depository. For example, human IgG4 constant region, human IgA1 constant region, human IgA2 constant region, human IgD constant region, human IgE constant region, and human IgM constant region are available at AAH25985, AAT74070, A2HU, P01880, AAB59424 and AAS01769, respectively.

In the present invention, therefore, the IgG4 Fc may comprise an amino acid sequence of SEQ ID NO: 1, the IgA1 Fc may comprise an amino acid sequence of SEQ ID NO: 2, IgA2 Fc may comprise an amino acid sequence of SEQ ID NO: 3, IgD Fc may comprise an amino acid sequence of SEQ ID NO: 4, IgE Fc may comprise an amino acid sequence of SEQ ID NO: 5, and IgM Fc may comprise an amino acid sequence of SEQ ID NO: 6.

In this regard, the region of native IgG4 Fc of SEQ ID NO: 1, which is replaced with a portion of CH2 domain of other Ig classes, may include all or a part of the FcγR binding site located at the N-terminus of CH2 domain of IgG4 Fc, and may include any region as long as binding of IgG4 Fc and FcγR can be inhibited to inhibit the ADCC-inducing ability.

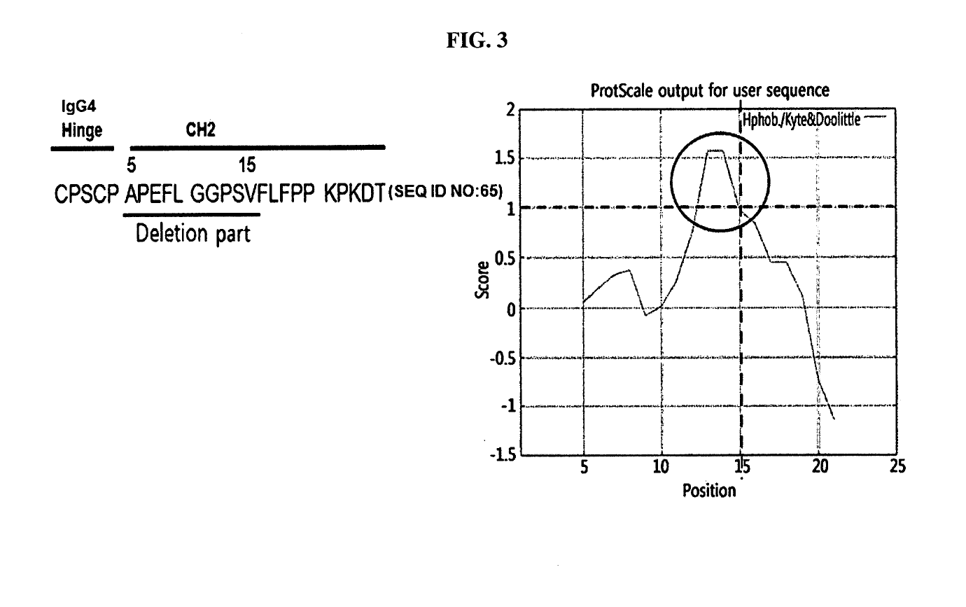

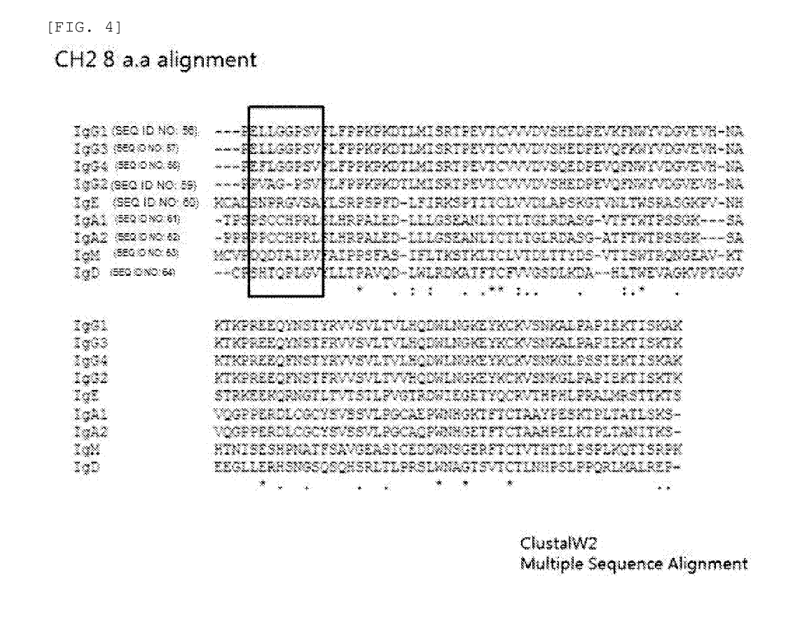

The CH2 domain of IgG4 Fc consists of amino acid residues at positions 111 to 220 of SEQ ID NO: 1. In one embodiment of the present invention, a region including FLGGPS sequence (SEQ ID NO: 55) (corresponding to amino acid residues at positions 114 to 119 of SEQ ID NO: 1) which is known to be the FcγR binding site in the CH2 domain of IgG4 Fc and 10 amino acid residues at the N-terminus having a hydrophobicity score of 1 or more were determined as a region to be removed by substitution (FIG. 3). Therefore, the N-terminal region of the CH2 domain of IgG4 Fc to be removed in the present invention is preferably at least three consecutive amino acid residues from the first amino acid residue in the FLGGPS sequence. Further, with respect to the objects of the present invention, 10 amino acid residues to be removed is replaced with amino acid residues of CH2 domain of an immunoglobulin of other different class. In this regard, it was confirmed that replacement of 8 amino acid residues is the most similar in terms of structural characteristic (FIG. 4). Accordingly, the amino acid residues of CH2 domain of an immunoglobulin of other different class to be inserted by replacement are preferably a sequence showing a high structural similarity with the amino acid residues to be removed, and more preferably, a sequence showing at least 50%, 60%, 70%, 80%, 90%, 91%, 92%, 93%, 94%, 95%, 96%, 97%, 98%, or 99% sequence identity therewith.

The region in IgG4 Fc of SEQ ID NO: 1 which is replaced in order to inhibit the ADCC-inducing ability, that is, a portion of the N-terminus of CH2 domain of IgG4 Fc to be removed by replacement may be preferably 6 to 20 consecutive amino acid residues in a direction from the position 111 to the C-terminus among the amino acid residues at positions 111 to 130 of SEQ ID NO: 1, more preferably, 6 to 14 consecutive amino acid residues in a direction from the position 111 to the C-terminus among the amino acid residues at positions 111 to 130 of SEQ ID NO: 1, and much more preferably, amino acid residues at positions 111 to 120 of SEQ ID NO: 1.

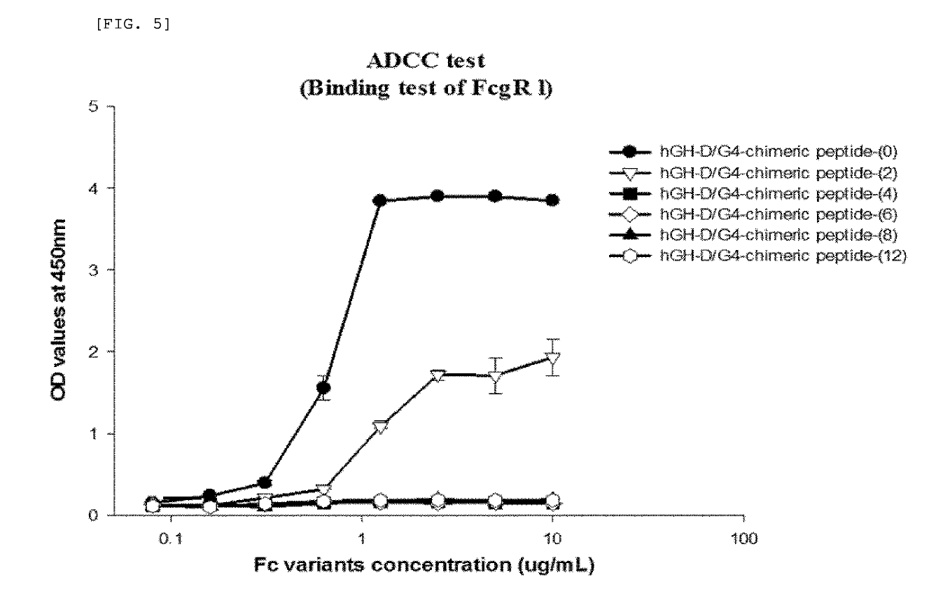

In another embodiment of the present invention, experiments were carried out by substituting the amino acid sequence of IgD Fc CH2 domain for amino acid sequences having different lengths in a direction from the amino acid residue at position 111 to the C-terminus. As a result, when only 4 amino acid residues were removed and replaced, the ADCC-inducing ability was maintained. However, when 6 to 14 amino acid residues were removed and replaced, the ADCC-inducing ability was completely eliminated. Therefore, it can be seen that removal of at least 6 amino acid residues is useful for elimination of the ADCC-inducing ability (FIG. 5).

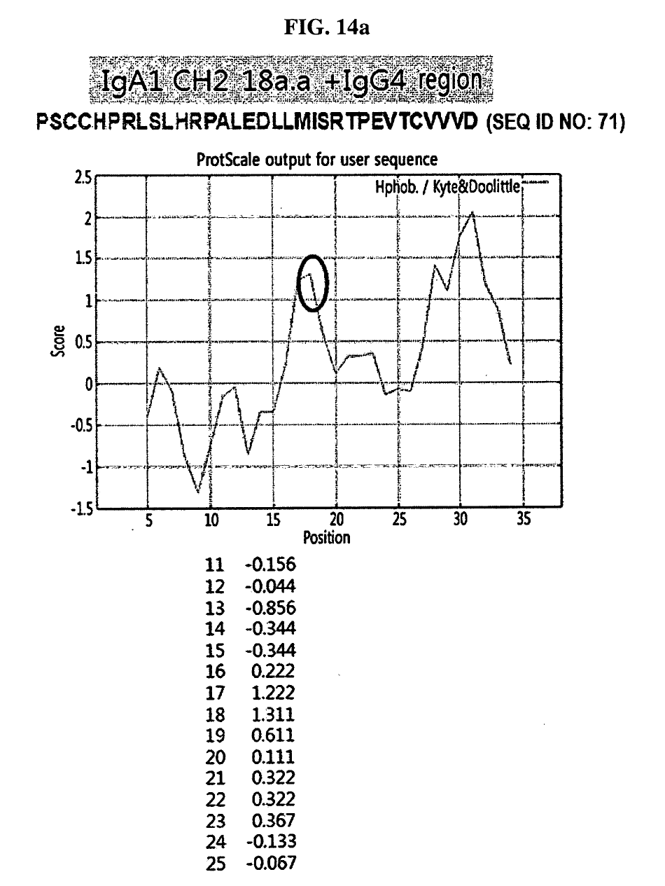

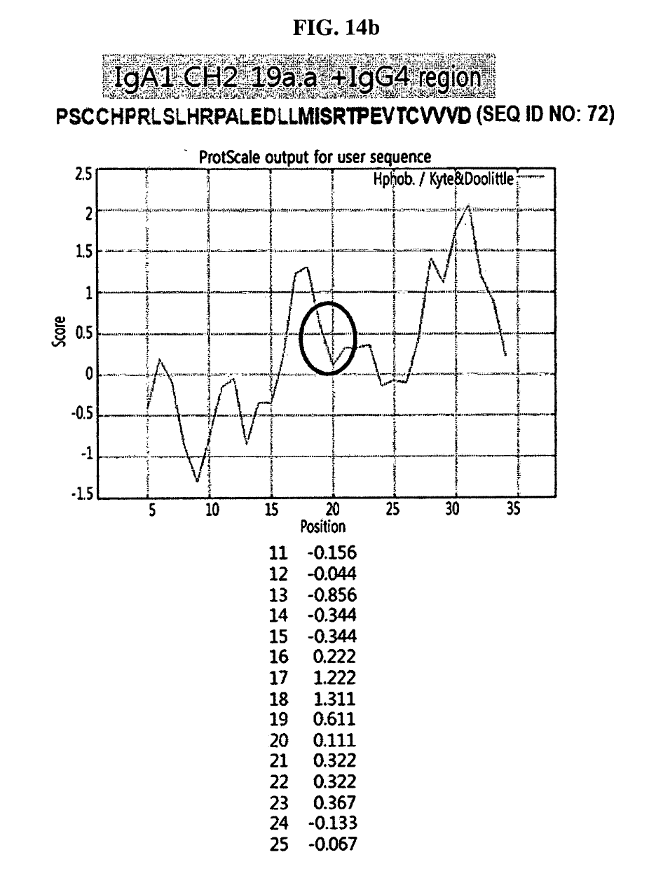

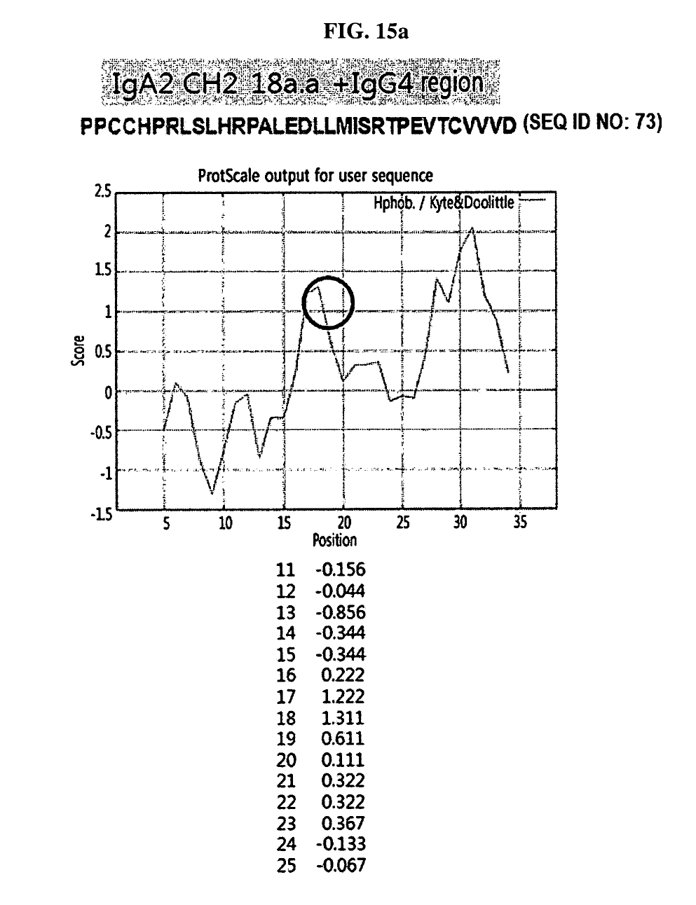

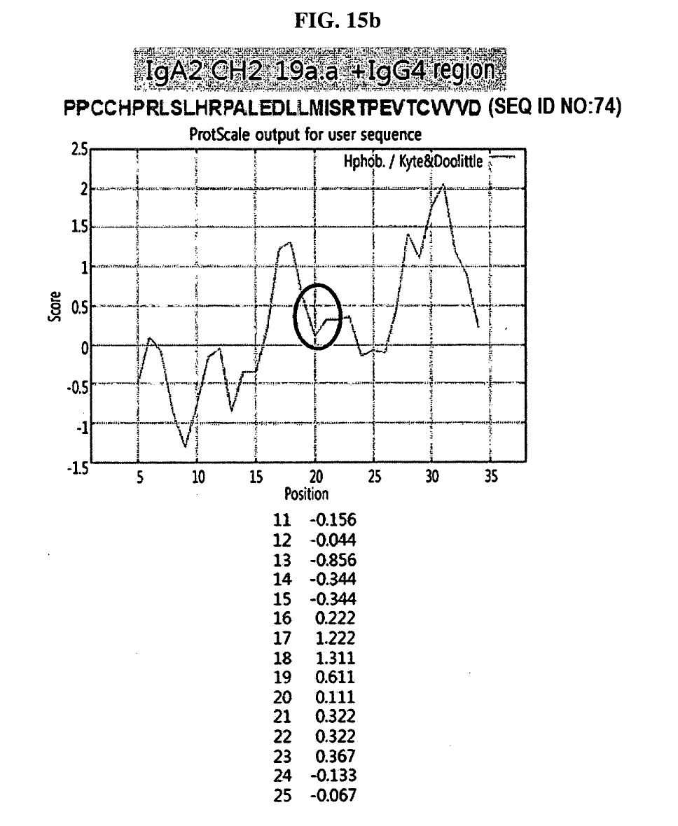

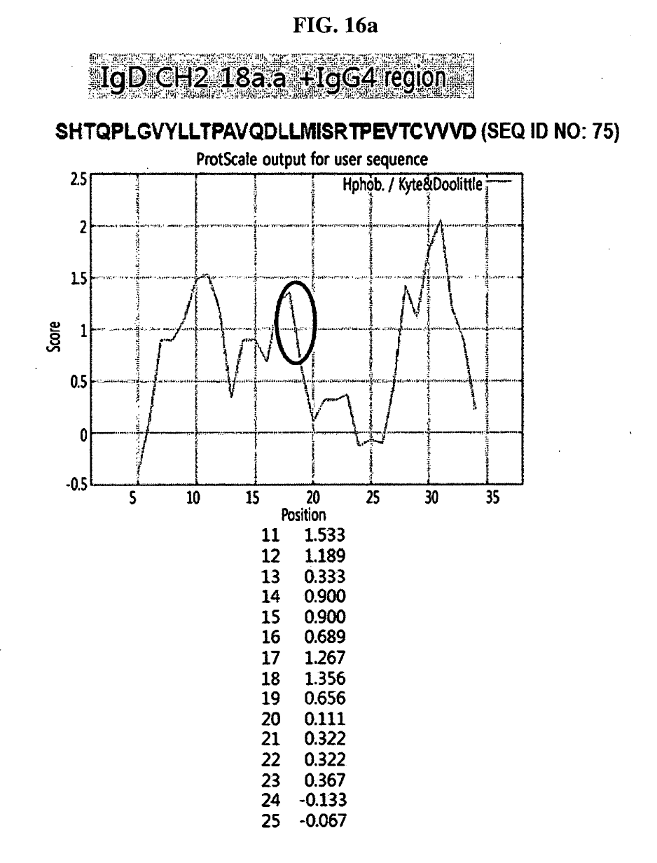

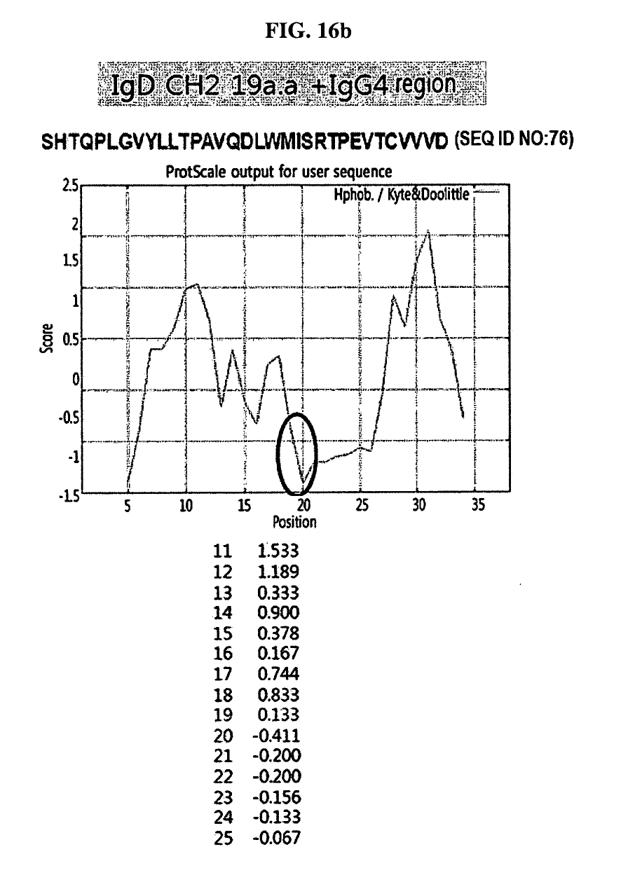

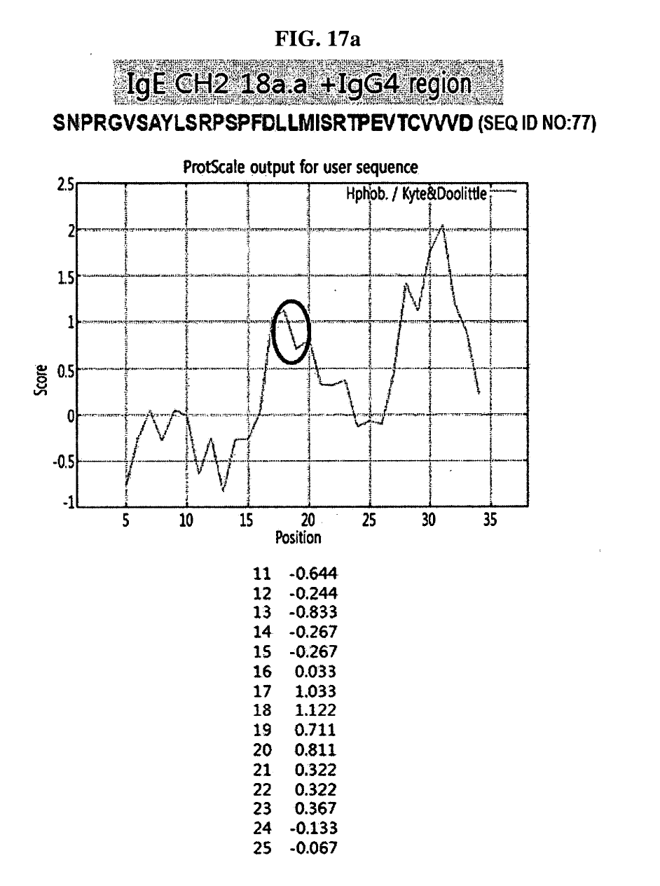

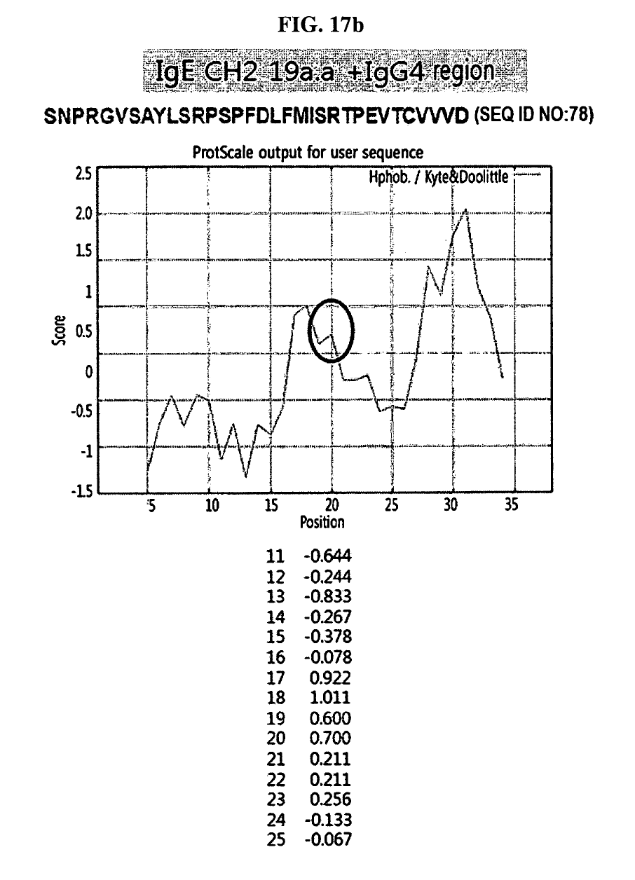

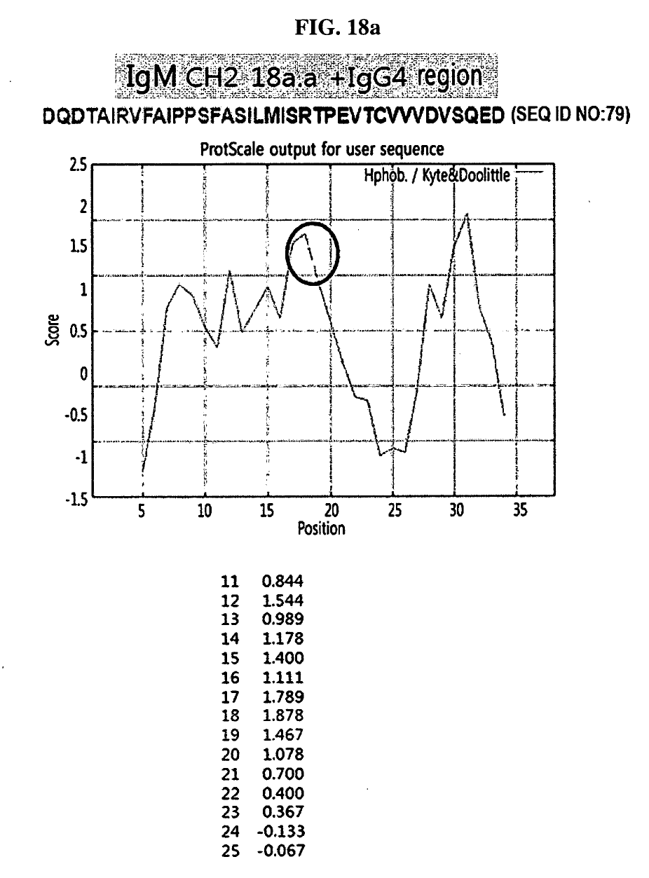

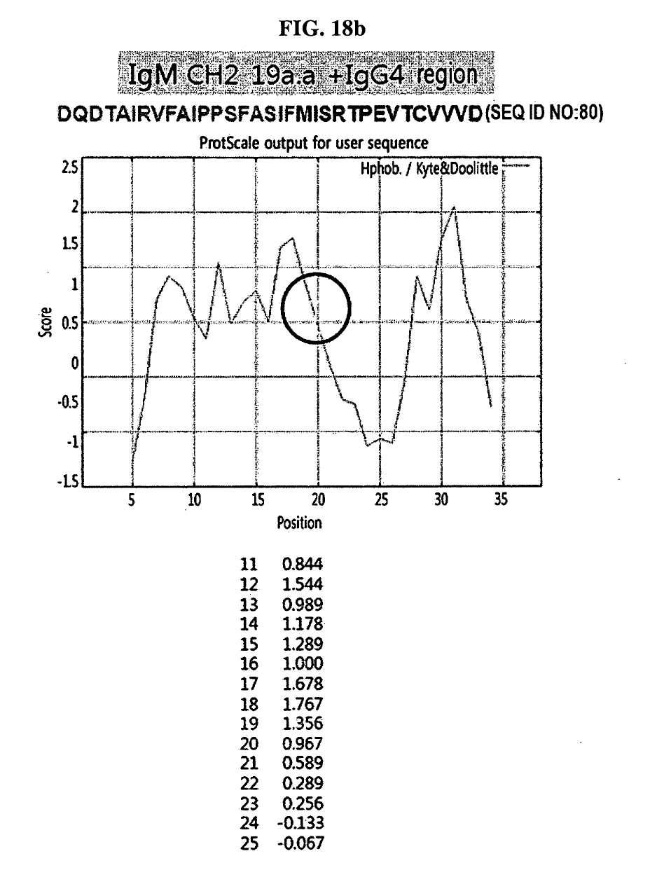

In still another embodiment of the present invention, hydrophobicity profiles were examined after 20 amino acid residues were removed and replaced with 18 amino acid residues, and 21 amino acid residues were removed and replaced with 19 amino acid residues. As a result, when 20 amino acid residues were removed and replaced with 18 amino acid residues, its hydrophobicity score was a high positive value. Thus, the junction site exists in the inner space to induce no undesirable immune responses. In contrast, when amino acid residues were removed and replaced with 19 amino acid residues, its hydrophobicity score was low. Thus, the junction site is exposed to the exterior upon formation of a three-dimensional structure, indicating a possibility of increasing immunogenicity. These results suggest that although up to 20 amino acid residues are removed and replaced, the same effect can be obtained (FIGS. 14a, 14b, 15a, 15b, 16a, 16b, 17a, 17b, 18a, and 18b).

The region which replaces the N-terminal portion of the CH2 domain of IgG4 Fc may be a portion of CH2 domain of IgA1, IgA2, IgD, IgE or IgM Fc. The amino acid sequences of CH2 domains of IgG1, IgG3, IgG4, IgG2, IgE, IgA1, IgA2, IgM, and IgD are shown as SEQ ID NOS: 56-64, respectively. The portion of CH2 domain of IgA1 Fc may be preferably a 4 to 18 consecutive amino acid sequence in a direction from the position 120 to the C-terminus among the amino acid residues at positions 120 to 137 of SEQ ID NO: 2, more preferably a 4 to 12 consecutive amino acid sequence in a direction from the position 120 to the C-terminus among the amino acid residues at positions 120 to 137 of SEQ ID NO: 2, and much more preferably, an amino acid sequence at positions 120 to 127 of SEQ ID NO: 2. Further, the portion of CH2 domain of IgA2 Fc may be preferably a 4 to 18 consecutive amino acid sequence in a direction from the position 107 to the C-terminus among the amino acid residues at positions 107 to 124 of SEQ ID NO: 3, more preferably a 4 to 12 consecutive amino acid sequence in a direction from the position 107 to the C-terminus among the amino acid residues at positions 107 to 124 of SEQ ID NO: 3, and much more preferably, an amino acid sequence at positions 107 to 114 of SEQ ID NO: 3. Further, the portion of CH2 domain of IgD Fc may be preferably a 4 to 18 consecutive amino acid sequence in a direction from the position 163 to the C-terminus among the amino acid residues at positions 163 to 180 of SEQ ID NO: 4, more preferably a 4 to 12 consecutive amino acid sequence in a direction from the position 163 to the C-terminus among the amino acid residues at positions 163 to 180 of SEQ ID NO: 4, and much more preferably, an amino acid sequence at positions 163 to 170 of SEQ ID NO: 4. Further, the portion of CH2 domain of IgE Fc may be preferably a 4 to 18 consecutive amino acid sequence in a direction from the position 208 to the C-terminus among the amino acid residues at positions 208 to 225 of SEQ ID NO: 5, more preferably a 4 to 12 consecutive amino acid sequence in a direction from the position 208 to the C-terminus among the amino acid residues at positions 208 to 225 of SEQ ID NO: 5, and much more preferably, an amino acid sequence at positions 208 to 215 of SEQ ID NO: 5. Further, the portion of CH2 domain of IgM Fc may be preferably a 4 to 18 consecutive amino acid sequence in a direction from the position 213 to the C-terminus among the amino acid residues at positions 213 to 230 of SEQ ID NO: 6, more preferably a 4 to 12 consecutive amino acid sequence in a direction from the position 213 to the C-terminus among the amino acid residues at positions 213 to 230 of SEQ ID NO: 6, and much more preferably, an amino acid sequence at positions 213 to 220 of SEQ ID NO: 6.

In one embodiment of the present invention, the portion of CH2 domain of IgG4 Fc was first intended to be replaced with a portion of CH2 domain of IgD Fc. When only 2 amino acid residues of IgD Fc CH2 domain were replaced and inserted, the ADCC-inducing ability was maintained. However, when 4 to 12 amino acid residues were replaced and inserted, the ADCC-inducing ability was completely eliminated. Therefore, it can be seen that replacement and insertion of at least 4 amino acid residues are useful for elimination of the ADCC-inducing ability (FIG. 5).

Further, in another embodiment of the present invention, hydrophobicity profiles of IgA1, IgA2, IgD, IgE and IgM Fc were examined after 20 amino acid residues were removed and replaced with 18 amino acid residues, and 21 amino acid residues were removed and replaced with 19 amino acid residues. As a result, when 20 amino acid residues were removed and replaced with 18 amino acid residues, their hydrophobicity scores were high positive values. Thus, the junction site exists in the inner space to induce no undesirable immune responses. In contrast, when 21 amino acid residues were removed and replaced with 19 amino acid residues, their hydrophobicity scores were low. Thus, the junction site is exposed to the exterior upon formation of a three-dimensional structure, indicating a possibility of increasing immunogenicity. These results suggest that although up to 18 amino acid residues are inserted, the same effect can be obtained (FIGS. 14a, 14b, 15a, 15b, 16a, 16b, 17a, 17b, 18a, and 18b).

In the replacement of the portion of CH2 domain, the number of amino acid residues of CH2 domain of IgG4 Fc to be removed and the number of amino acid residues of CH2 domain of IgA1, IgA2, IgD, IgE or IgM Fc to be inserted are the same as or different from each other. However, in order to maintain the basic structure of immunoglobulin Fc and to prevent unexpected side effects, it is advantageous that a difference in the number between the amino acid residues to be removed and inserted is small. Preferably, the number of the amino acid residues to be removed and the number of the amino acid residues to be inserted are the same or a difference therebetween is 4 or less, or 2 or less. More preferably, 2 amino acid residues can be further removed and amino acid residues cannot be further removed. In one embodiment of the present invention, 10 amino acid residues were removed from CH2 domain of IgG4 Fc, and 8 amino acid residues of CH2 domain of IgA1, IgA2, IgD, IgE or IgM Fc were inserted, and then the efficacy was examined.

In the present invention, since the regions other than the CH2 domain, that is, the hinge region and the CH3 domain have less influence on the increase of half-life and the ADCC-inducing ability, they may have any sequences derived from various immunoglobulins as long as they do not alter the structure or function of the polypeptide of the present invention.

In the present invention, the hinge region functions to maintain its structure by maintaining flexibility when it binds with the biologically active molecule. The hinge region may be any hinge region of all immunoglobulins, as long as it does not alter the function of the polypeptide, that is, its long half-life is maintained and ADCC-inducing ability is eliminated. For example, the hinge region may be a hinge region of human IgG1, IgG2, IgG3, IgG4, IgA1, IgA2, IgD, IgE or IgM, preferably, a hinge region of human IgG4, IgA1, IgA2, IgD, IgE or IgM, which is included as a component of the polypeptide, and more preferably, a human IgG4 hinge region or a human IgD hinge region. Further, the human IgG4 hinge region is composed of amino acid residues at positions 99 to 110 of SEQ ID NO: 1, and the hinge region included in the polypeptide of the present invention may be preferably a 5 to 12 consecutive amino acid sequence in a direction from the position 110 to the N-terminus among the amino acid residues at positions 99 to 110 of SEQ ID NO: 1, and more preferably, the amino acid residues at positions 99 to 110 of SEQ ID NO: 1. Further, the human IgD hinge region is composed of amino acid residues at positions 99 to 162 of SEQ ID NO: 4, and the hinge region included in the polypeptide of the present invention may be preferably a 5 to 64 consecutive amino acid sequence in a direction from the position 162 to the N-terminus among the amino acid residues at positions 99 to 162 of SEQ ID NO: 4, and more preferably, an amino acid sequence at positions 133 to 162 of SEQ ID NO: 4.

Further, the hinge region of the present invention binds with the biologically active molecule to maintain the structure and activity of a chimeric polypeptide. In this regard, it may be more advantageous that the junction site having a predetermined length or longer is included. The hinge region of human IgG4 is relatively short, compared to the hinge region of IgD, and thus a linker may be further linked to the N-terminus of IgG4 hinge region when it binds with the biologically active molecule.

Further, the hinge region of the present invention may include amino acid mutations for preventing its cleavage, and for example, it may include amino acid mutations of substitution of K (lysine) at position 144 of SEQ ID NO: 4 with G (Glycine) and E (Glutamic acid) at position 145 of SEQ ID NO: 4 with G (Glycine) or S (Serine), but is not limited thereto.

In the present invention, the CH3 domain may be any region of CH3 domain of IgG4 Fc, as long as it does not alter the function of the polypeptide, that is, the ADCC-inducing ability can be removed while maintaining its long half-life. The CH3 domain of IgG4 Fc is composed of amino acid residues at positions 221 to 327 of SEQ ID NO: 1, preferably an 80 to 107 consecutive amino acid sequence in a direction from the position 221 to the C-terminus among the amino acid residues at positions 221 to 327 of SEQ ID NO: 1, and more preferably, the amino acid residues at positions 221 to 327 of SEQ ID NO: 1.

The polypeptide may further include a CH1 domain, and the CH1 domain may binds to the N-terminus of the hinge region. The CH1 domain may be a CH1 domain of any human immunoglobulin, as long as it does not alter the function of the polypeptide. For example, the CH1 domain may be a CH1 domain of human IgG1, IgG2, IgG3, IgG4, IgA1, IgA2, IgD, IgE or IgM, and preferably, the CH1 domain of human IgG4.

Based on the above description, the polypeptide of the present invention may be represented by the following Formula:

N′—Y—Z2-Z3-Z4-C′

wherein N′ is the N-terminus of the polypeptide and C′ is the C-terminus thereof;

Y is a hinge region;

Z2 is a portion of the N-terminus of human IgA1 Fc CH2 domain, human IgA2 Fc CH2 domain, human IgD Fc CH2 domain, human IgE Fc CH2 domain or IgM Fc CH2 domain;

Z3 is a portion of the C-terminus of human IgG4 Fc CH2 domain, in which a portion of the N-terminus is removed; and

Z4 is a human IgG4 Fc CH3 domain.

Further, the polypeptide of the present invention may have the following form by additionally linking a CH1 domain or a linker to the N-terminus of the hinge region:

N′—(Z1)ₙ-Y—Z2-Z3-Z4-C′

N′-(L)ₘ-Y—Z2-Z3-Z4-C′

wherein Z1 is a CH1 domain region;

L is a linker;

n is an integer of 0 or 1; and

m is an integer of 0 or 1.

The following Table 1 represents a preferred amino acid sequence of each region of Ig fragment.

TABLE 1

| | Preferred range | Sequence of longest | SEQ | Position in |
|---|---|---|---|---|
| Fc domain | of Ig fragment | fragment within preferred range | ID NO: | SEQ ID NO. |
| Hinge | 5-12 C-terminal | ESKYGPPCPSCP | 45 | 99-110 of |
| region | amino acid residues | | | SEQ ID NO: 1 |
| (Y) | of IgG4 hinge region | | | |
| | 5-64 C-terminal | RWPESPKAQASSVPTAQPQAEGSLAKATTAPA | 46 | 99-162 of |
| | amino acid residues | TTRNTGRGGEEKKKEKEKEEQEERETKTPECP | | SEQ ID NO: 4 |
| | of IgD hinge region | | | |
| N-terminus | 4-18 N-terminal | PSCCHPRLSLHRPALEDL | 47 | 120-137 of |
| of CH2 | amino acid residues | | | SEQ ID NO: 2 |
| domain | of IgA1 CH2 | | | |
| (Z2) | 4-18 N-terminal | PPCCHPRLSLHRPALEDL | 48 | 107-124 of |
| | amino acid residues | | | SEQ ID NO: 3 |
| | of IgA2 CH2 | | | |
| | 4-18 N-terminal | SHTQPLGVYLLTPAVQDL | 49 | 163-180 of |
| | amino acid residues | | | SEQ ID NO: 4 |
| | of IgD CH2 | | | |
| | 4-18 N-terminal | SNPRGVSAYLSRPSPFDL | 50 | 208-225 of |
| | amino acid residues | | | SEQ ID NO: 5 |
| | of IgE CH2 | | | |
| | 4-18 N-terminal | DQDTAIRVFAIPPSFASI | 51 | 213-230 of |
| | amino acid residues | | | SEQ ID NO: 6 |
| | of IgM CH2 | | | |
| C-terminus | 90-104 C-terminal | GPSVFLFPPKPKDTLMISRTPEVTCVVVDVSQ | 52 | 117-220 of |
| of CH2 | amino acid residues | EDPEVQFNWYVDGVEVHNAKTKPREEQFNSTY | | SEQ ID NO: 6 |
| domain | of IgG4 CH2 | RVVSVLTVLHQDWLNGKEYKCKVSNKGLPSSI | | |
| (Z3) | | EKTISKAK | | |
| CH3 | 80-107 N-terminal | GQPREPQVYTLPPSQEEMTKNQVSLTCLVKGF | 53 | 221-327 of |
| domain | amino acid residues | YPSDIAVEWESNGQPENNYKTTPPVLDSDGSF | | SEQ ID NO: 1 |
| (Z4) | of IgG4 CH3 | FLYSRLTVDKSRWQEGNVFSCSVMHEALHNHY | | |
| | | TQKSLSLSLGK | | |
| CH1 | IgG4 CH1 | PSVFPLALCSRSTSESTAALGCLVKDYFPEP | 54 | |
| domain | | VTVSWNSGALTSGVHTFPAVLQSSGLYSLSS | | |
| (Z1) | | VVTVPSSSLGTKTYTCNVDHKPSNTKVDKRV | | |

In the above Table, the underlined region represents the shortest fragment within the preferred range of amino acid residues.

In one preferred specific embodiment, the polypeptide may preferably comprise an amino acid sequence of SEQ ID NO: (X-L/A1/G4), SEQ ID NO: 10 (X-L/A2/G4), SEQ ID NO: 11 (X-L/D/G4), SEQ ID NO: 12 (X-L/E/G4), SEQ ID NO: 13 (X-L/M/G4) which has an IgG4 hinge region as a hinge region, and preferably comprise an amino acid sequence of SEQ ID NO: 14(X-D/A1/G4), SEQ ID NO: 15(X-D/A2/G4), SEQ ID NO: 16(X-D/E/G4) and SEQ ID NO: 17(X-D/M/G4) which has an IgD hinge region as a hinge region.

In another aspect, the present invention provides a chimeric polypeptide including the polypeptide and the biologically active molecule. The chimeric polypeptide is formed by fusion of the above described Fc polypeptide and the biologically active molecule (biologically active protein, biologically active polypeptide, polypeptide drug), and in the present invention, the “Fc fusion polypeptide”, “biologically active molecule-Fc fusion protein” or “fusion protein” can be used interchangeably.

When the above described polypeptide of the present invention binds with the biologically active molecule, it shows the effects of increasing the serum half-life of the biologically active molecule and the expression level thereof to optimize the activity thereof, and also, the ADCC-inducing ability can be eliminated. Therefore, when a chimeric polypeptide prepared by conjugation of the polypeptide with the biologically active molecule is provided, many advantages can be obtained.

The biologically active molecule may be fused to the N-terminus or C-terminus of the polypeptide, and the resulting chimeric polypeptide is able to show increased circulating half-life, compared to the native circulating half-life of the biologically active molecule. Further, the biologically active molecule may be preferably fused to the N-terminus of the polypeptide via a linker.

The linker may be a peptide linker, which is composed of 1 to 50 amino acid residues. Preferably, the linker may be a peptide linker of 10 to 20 amino acid residues composed of Gly and Ser residues, and more preferably, a linker of GGGGSGGGGSGGGGS (SEQ ID NO: 7).

When the linker is used, the linker and a polypeptide drug may be prepared by a specific method. That is, the linker may be linked to the N-terminus, the C-terminus or a free group of the Fc fragment, and may also be linked to the N-terminus, the C-terminus or a free group of the polypeptide drug. When the linker is a peptide linker, the linkage may take place at a certain linking site. When the polypeptide drug and the Fc polypeptide are expressed separately and then joined to each other, the coupling may be performed using any of a number of coupling agents known in the art. Examples of the coupling agents include 1,1-bis(diazoacetyl)-2-phenylethane, glutaradehyde, N-hydroxysuccinimide esters such as 4-azidosalicylic acid, imidoesters including disuccinimidyl esters such as 3,3′-dithiobis(succinimidylpropionate), and bifunctional maleimides such as bis-N-maleimido-1,8-octane, but are not limited thereto.

The biologically active molecule binding to the polypeptide of the present invention, that is, the biologically active molecule included as one component of the chimeric polypeptide may be a soluble protein. Specifically, it may be a hormone, cytokine, growth factor, co-stimulatory molecule, hormone receptor, cytokine receptor, growth factor receptor, or short peptide, but is not limited thereto. For example, the biologically active protein may be GM-CSF, IL-1, IL-2, IL-3, IL-4, IL-5, IL-6, IL-7, IL-8, IL-10, IL-10 receptor, TGF-β, TGF-β receptor, IL-17, IL-17 receptor, Factor VII, CSCL-11, FSH, human growth hormone, BMP-1 (bone morphogenetic protein-1), CTLA4, PD-1, PD-L1, PD-L2, GLP-1, betacellulin, OPG, RNAK, interferon-alpha, interferon-beta or their variants/fragments. It may also include, but is not limited to, a Fab region of an antibody. The biologically active molecule may be also a secreted protein.

As used herein, the term “variant” refers to a polynucleotide or nucleic acid differing from a reference nucleic acid or polypeptide, but retaining essential properties thereof. Generally, variants are overall closely similar, and, in many regions, identical to the reference nucleic acid or polypeptide. Also, the term “variant” refers to a biologically active portion of a biologically active molecule drug, which retains at least one functional and/or therapeutic property thereof as described elsewhere herein or otherwise known in the art. Generally, variants are overall very similar, and, in many regions, identical to the amino acid sequence of the biologically active polypeptide of interest.

Examples of biologically active protein drug capable of binding to the polypeptide of the present invention include human growth hormone, BMP-1 (bone morphogenetic protein-1), growth hormone releasing hormone, growth hormone releasing peptide, interferons and interferon receptors (e.g., interferon-α, -β and -γ, water-soluble type I interferon receptor, etc.), G-CSF (granulocyte colony stimulating factor), GM-CSF (granulocyte-macrophage colony stimulating factor), glucagon-like peptides (e.g., GLP-1, etc.), G-protein-coupled receptor, interleukins (e.g., interleukin-1, -2, -3, -4, -5, -6, -7, -8, -9, -10, -11, -12, -13, -14, -15, -16, -17, -18, -19, -20, -21, -22, -23, -24, -25, -26, -27, 28, -29, -30, etc.) and interleukin receptors (e.g., IL-1 receptor, IL-4 receptor, etc.), enzymes (e.g., glucocerebrosidase, iduronate-2-sulfatase, alpha-galactosidase-A, agalsidase alpha and beta, alpha-L-iduronidase, butyrylcholinesterase, chitinase, glutamate decarboxylase, imiglucerase, lipase, uricase, platelet-activating factor acetylhydrolase, neutral endopeptidase, myeloperoxidase, etc.), interleukin and cytokine binding proteins (e.g., IL-18 bp, TNF-binding protein, etc.), macrophage activating factor, macrophage peptide, B cell factor, T cell factor, protein A, allergy inhibitor, cell necrosis glycoproteins, immunotoxin, lymphotoxin, tumor necrosis factor, tumor suppressors, metastasis growth factor, alpha-1 antitrypsin, albumin, alpha-lactalbumin, apolipoprotein-E, erythropoietin, highly glycosylated erythropoietin, angiopoietins; hemoglobin, thrombin, thrombin receptor activating peptide, thrombomodulin, factor VII, factor VIIa, factor VIII, factor IX, factor XIII, plasminogen activating factor, fibrin-binding peptide, urokinase, streptokinase, hirudin, protein C, C-reactive protein, renin inhibitor, collagenase inhibitor, superoxide dismutase, leptin, platelet-derived growth factor, epithelial growth factor, epidermal growth factor, angiostatin, angiotensin, bone growth factor, bone stimulating protein, calcitonin, insulin, atriopeptin, cartilage inducing factor, elcatonin, connective tissue activating factor, tissue factor pathway inhibitor, follicle stimulating hormone, luteinizing hormone, luteinizing hormone releasing hormone, nerve growth factors (e.g., nerve growth factor, ciliary neurotrophic factor, AF-1 (axogenesis factor-1), brain-natriuretic peptide, glial derived neurotrophic factor, netrin, neurophil inhibitor factor, neurotrophic factor, neuturin, etc.), parathyroid hormone, relaxin, secretin, somatomedin, insulin-like growth factor, adrenocortical hormone, glucagon, cholecystokinin, pancreatic polypeptide, gastrin releasing peptide, corticotropin releasing factor, thyroid stimulating hormone, autotaxin, lactoferrin, myostatin, receptors (e.g., TNFR(p75), TNFR(p55), IL-1 receptor, VEGF receptor, B cell activating factor receptor, etc.), receptor antagonists (e.g., IL1-Ra etc.), cell surface antigens (e.g., CD2, 3, 4, 5, 7, 11a, 11b, 18, 19, 20, 23, 25, 33, 38, 40, 45, 69, etc.), virus vaccine antigens, monoclonal antibodies, polyclonal antibodies, antibody fragments (e.g., scFv, Fab, Fab′, F(ab′)2 and Fd), and virus derived vaccine antigens. An antibody fragment may be Fab, Fab′, F(ab′)2, Fd or scFv, which is capable of binding to a specific antigen, and preferably Fab′. The Fab fragments contain the variable domain (VL) and constant domain (CL) of the light chain and the variable domain (VH) and the first constant domain (CH1) of the heavy chain. The Fab′ fragments differ from the Fab fragments in terms of adding several amino acid residues including one or more cysteine residues from the hinge region to the carboxyl terminus of the CH1 domain. The Fd fragments includes only the VH and CH1 domain, and the F(ab′)2 fragments are produced as a pair of Fab′ fragments by either disulfide bonding or a chemical reaction. The scFv (single-chain Fv) fragments include the VL and VH domains that are linked to each other by a peptide linker and thus are present in a single polypeptide chain.

In one embodiment of the present invention, human Growth Hormone (hGH) was used as the biologically active molecule to examine the efficacy of the chimeric polypeptide of the present invention.

Based on the above description, the chimeric polypeptide of the present invention may be represented by the following Formula:

X-(L)ₙ-(Z1)ₘ-Y—Z2-Z3-Z4

wherein X is a biologically active molecule;

Y is a hinge region of IgG4 of IgD;

Z2-Z3 is the modified CH2 domain of IgG4, wherein Z2 is one selected from the group consisting of a 4-18 consecutive amino acid sequence starting from position 1 of SEQ ID NO: 47, a 4-18 consecutive amino acid sequence starting from position 1 of SEQ ID NO: 48, a 4-18 consecutive amino acid sequence starting from position 1 of SEQ ID NO: 49, a 4-18 consecutive amino acid sequence starting from position 1 of SEQ ID NO: 50, and a 4-18 consecutive amino acid sequence starting from position 1 of SEQ ID NO: 51; and Z3 is a 90-104 consecutive amino acid sequence starting from the C-terminus of SEQ ID NO: 52, wherein the total length of the Z2-Z3 may be 94-122 amino acid residues;

Z4 is a human IgG4 Fc CH3 domain;

L is a linker;

Z1 is a CH1 domain;

m is an integer of 0 or 1; and

n is an integer of 0 or 1.

In another embodiment, the total length of the Z2-Z3 may be 94-166 amino acid residues.

In one preferred specific embodiment, the chimeric polypeptide may have preferably an amino acid sequence of SEQ ID NO: 18 (hGH-L/A1/G4), SEQ ID NO: 19 (hGH-L/A2/G4), SEQ ID NO: 20 (hGH-L/D/G4), SEQ ID NO: 21 (hGH-L/E/G4), or SEQ ID NO: 22 (hGH-L/M/G4) in which hGH as the biologically active molecule and IgG4 hinge region as the hinge region are linked via a linker, and an amino acid sequence of SEQ ID NO: 23 (hGH-D/A1/G4), SEQ ID NO: 24 (hGH-D/A2/G4), SEQ ID NO: 25 (hGH-D/E/G4) or SEQ ID NO: 26 (hGH-D/M/G4) in which hGH is used as the biologically active molecule and IgD hinge region is used as the hinge region. In this regard, hGH used as the biologically active molecule is in the form in which a signal sequence at the N-terminus is removed, and a non-removal form can be also included in the scope of the present invention.

In still another aspect of the present invention, the present invention provides a method for producing the polypeptide or the chimeric polypeptide. Specifically, the method may include the steps of (i) introducing a nucleic acid molecule coding for the polypeptide or the chimeric polypeptide into a mammalian host cell, (ii) culturing the cell under conditions where the polypeptide or the chimeric polypeptide can be expressed; and (iii) harvesting the expressed polypeptide or the chimeric polypeptide.

In the present invention, the chimeric polypeptide functions as a polypeptide drug while retaining the above described usefulness. The chimeric polypeptide can be produced by preparing a construct including the nucleic acid molecule encoding the chimeric polypeptide, expressing the construct in a host cell, and then harvesting the chimeric polypeptide. At this time, any typical method known in the art can be used for the production. According to circumstances, the chimeric polypeptide can be produced by expressing a nucleotide encoding the Fc polypeptide and then binding it to the biologically active molecule according to the typical method. The mammalian host cell may be CHO, COS, CAPTI or BHK cell.

In still another aspect, the present invention provides an isolated nucleic acid molecule encoding the polypeptide or the chimeric polypeptide, an expression vector including the nucleic acid molecule, and a host cell including the expression vector.

The nucleic acid molecule encoding the polypeptide of the present invention may preferably encode a polypeptide having an amino acid sequence of SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 or SEQ ID NO: 17, and it may more preferably comprise a nucleotide sequence of SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34 or SEQ ID NO: 35. Further, the nucleic acid molecule encoding the chimeric polypeptide of the present invention may preferably encode a chimeric polypeptide having an amino acid sequence of SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 26, and it may comprise a nucleotide sequence of SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43 or SEQ ID NO: 44.

Due to the codon degeneracy or in consideration of the codons preferred by an organism where the polypeptide (chimeric polypeptide) is expressed, the nucleic acid molecule or the polynucleotide may contain various alterations as long as the amino acid sequence of the polypeptide (chimeric polypeptide) to be expressed is not changed.

Hereinafter, the present invention will be described in more detail with reference to Examples. However, these Examples are for illustrative purposes only, and the invention is not intended to be limited by these Examples.

### Example 1

### Test of In-Vivo Half-Life of Wild-Type IgG4 Fc Fusion Protein

First, the effect of fusion of the wild-type IgG4 Fc to a biologically active protein on in-vivo half-life of the biologically active protein was examined.

In detail, human growth hormone (hGH, NP_000506) was used as the biologically active protein, and a commercially available growth hormone, somatropin (recombinant human growth hormone) was used as a control group. To investigate the pharmacokinetics thereof, the following experiment was carried out.

Each protein (hGH-IgG4Fc-wt as an experimental group and somatropin as a control group) was administered via SC (subcutaneous) route to 4 male Sprague Dawley rats per group. Blood was obtained before injection and 2, 6, 12, 24, 48, 72, 96, 120, 144 and 168 h post-injection. The blood samples were incubated at room temperature for 30 minutes for coagulation, and then sera were obtained by centrifugation at 3000 rpm for 10 minutes and stored at a deep freezer. For quantification, samples were diluted using an hGH kit (Roche, Cat#11585878001) so that each dilution of the standard curve falls on a straight line.

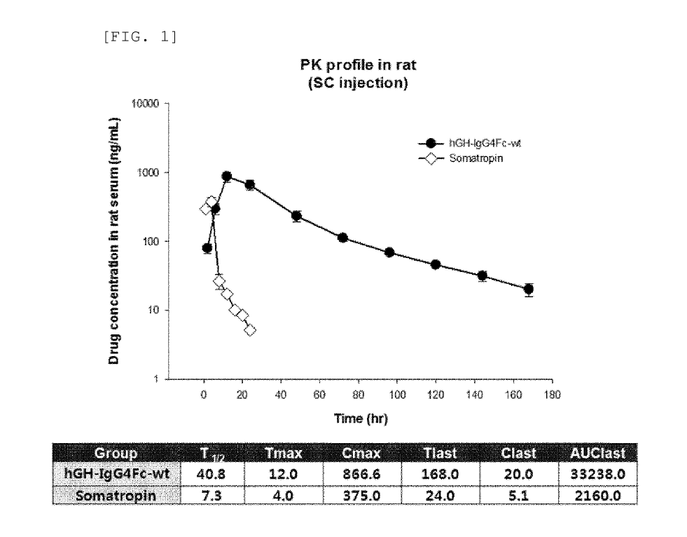

As shown in FIG. 1, somatropin as a single hGH which is not fused with the Fc protein showed 7.3 hr of half-life, whereas the fusion protein (hGH-IgG4Fc-wt) of hGH and the wild-type IgG4 Fc showed an about 5.5-fold increase in the half-life.

### Example 2

### FcγR Binding Assay of Wild-Type IgG4 Fc Fusion Protein

In order to investigate whether hGH-IgG4Fc-wt fusion protein having the increased in-vivo half-life by fusion of IgG4Fc-wt in Example 1 binds to FcγR to induce ADCC, the following experiment was carried out.

In this regard, IgG1 has a long in-vivo half-life of 21 days, like IgG4, but it binds to FcγR to induce ADCC. Thus, rituxan which is an IgG1 antibody against CD20 was used as a positive control.

In detail, two-fold dilution of each protein (hGH-IgG4Fc-wt and rituxan) was carried out from 10 μg/ml to 80 ng/ml, and each 100 μl thereof was dispensed to a 96-well plate and allowed to bind at 4° C. overnight. The protein-coated plate was washed with a washing buffer (PBS containing 0.05% tween) and blocked using a blocking buffer (PBS containing 1% BSA) at room temperature for 1 hour, and then 2 μg/ml of FcγRI (R&D cat#BAF1257) was added to each well, and allowed to react at room temperature for 2 hours. After reaction, all wells were washed with the washing buffer. To investigate FcγRI binding, 2 μg/ml of biotinylated anti-FcγRI (R&D, cat#1257-FC) was added to each well, and then allowed to react at room temperature for 1 hour. After reaction, the plate was washed with the washing buffer, and then 3000-fold diluted streptavidin-HRP (BD, cat#554066) was added to each well, and then allowed to react at room temperature for 30 minutes under dark condition. After reaction, the plate was washed with the washing buffer, and TMB as a substrate of peroxidase was added and 2N H₂SO₄ was added to terminate the reaction. Absorbance was examined at 450 nm/570 nm.

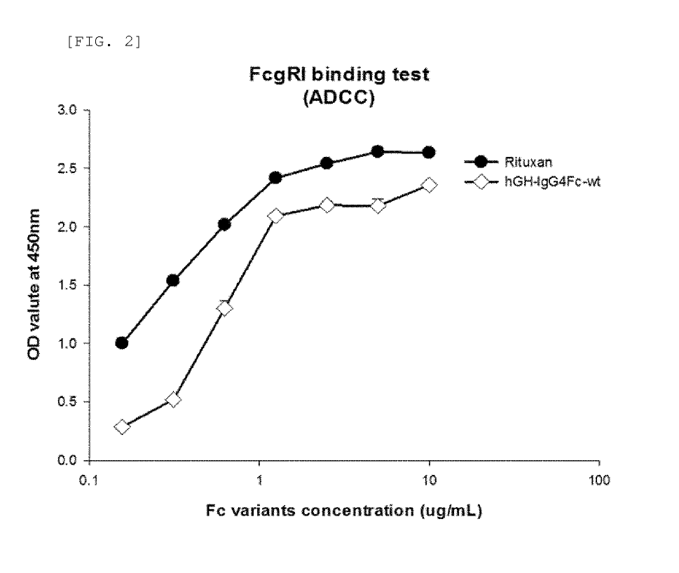

As shown in FIG. 2, the protein (hGH-IgG4Fc-wt) fused with the wild-type IgG4 Fc showed a strong binding to FcγRI, like the positive control rituxan, indicating the function of inducing ADCC.

### Example 3

### FcγR Binding Site of CH2 Domain of Wild-Type IgG4 Fc and Replacement with Immunoglobulin CH2 Domain of Different Class

As confirmed in Examples 1 and 2, the wild-type IgG4 Fc has the efficacy of increasing in-vivo half-life of the biologically active protein fused thereto, but it strongly binds to FcγR to induce ADCC, leading to a side effect of cytotoxicity. Therefore, for practical use, it is required to solve this problem.

Accordingly, to investigate FcγR binding site of the wild-type IgG4 Fc domain and to solve the problem, the present inventors replaced the FcγR binding site of the wild-type IgG4 Fc domain with a portion of other immunoglobulin having no FcγR binding site to examine whether the ADCC-inducing ability of IgG4 can be inhibited.

Therefore, based on the highly hydrophobic region including the FcγR binding site of the wild-type IgG4 Fc domain, a region similar to the region to be removed from the wild-type IgG4 Fc domain was selected from the sequence of a different class of immunoglobulin having no FcγR binding site. Through this procedure, it was intended to develop an IgG4 variant which does not bind to FcγR and has a minimized immunogenicity-inducing ability with less modification of the structure of the wild-type IgG4 Fc domain.

First, referring to the literature (Current Opinion in Biotechnology 2009, 20:685-691), a region containing 10 N-terminal amino acids was selected as a reference sequence to be removed from CH2 domain of the wild-type IgG4 Fc, based on the region of the wild-type IgG4 Fc domain which has FLGGPS sequence known to be important in FcγR binding and has a hydrophobicity score of 1 or more (FIG. 3).

Thereafter, based on 10 amino acids removed from the N-terminus of CH2 domain of the wild-type IgG4 Fc, a structurally similar sequence was selected from each CH2 domain sequence of IgD, IgA1, IgA2, IgE and IgM using a ClustalW2 Multiple Sequence Alignment program (FIG. 4). As a result, it was found that replacement of 8 amino acids of CH2 domain of different class of immunoglobulin showed the most similar structure feature to replacement of 10 amino acids of CH2 domain of the wild-type IgG4 Fc.

Next, based on 10 amino acids to be removed from the N-terminus of CH2 domain of the wild-type IgG4 Fc and 8 amino acids of CH2 domain of different class of immunoglobulin to be inserted thereto, various constructs were prepared by switching a portion of the N-terminus of the wild-type IgG4 Fc domain with CH2 domain of IgD, and their binding to FcγRI was examined in order to investigate a minimal region having an influence on FcγR binding.

In detail, hGH-chimeric IgG4 variants were prepared by replacing 4, 6, 8, 10 and 14 amino acids at the N-terminus of IgG4 Fc CH2 domain with 2, 4, 6, 8 and 12 amino acids of IgD CH2 domain, respectively and the experiments were carried out using them in the same manner as in Example 2.

As shown in FIG. 5, the FcγRI binding ability of the variant prepared by replacing the portion of the IgG4 CH2 domain with 2 amino acids of IgD domain was decreased to half of the FcγRI binding ability of the wild-type hGH-IgG4Fc-wt having no mutation, but the variant maintained its FcγRI binding ability at a predetermined level. The FcγRI binding ability of the variant prepared by replacement with 4, 6, 8 or 12 amino acids of IgD CH2 domain was completely eliminated, and thus no ADCC was induced.

Taken together, 6 amino acids at the N-terminus of IgG4 CH2 domain is a minimal region required for FcγRI binding, and switching of this region with amino acids of different class, that is, amino acids of IgD CH2 domain showed no FcγRI binding to induce no ADCC, thereby avoiding the side effect of IgG4.

### Example 4

### Test of In-Vivo Half-Life of Protein Fused with CH2 Variant Prepared by Replacement of a Portion of N-Terminus of CH2 Domain of Wild-Type IgG4 Fc

As confirmed in Example 3, ADCC-including ability can be inhibited by switching of a portion of the N-terminus of CH2 domain of the wild-type IgG4 with amino acids of different class. Additionally, its effect on in-vivo half-life was examined.

To achieve this, the experiment was carried out using the variants prepared in Example 3 in the same manner as in Example 1.

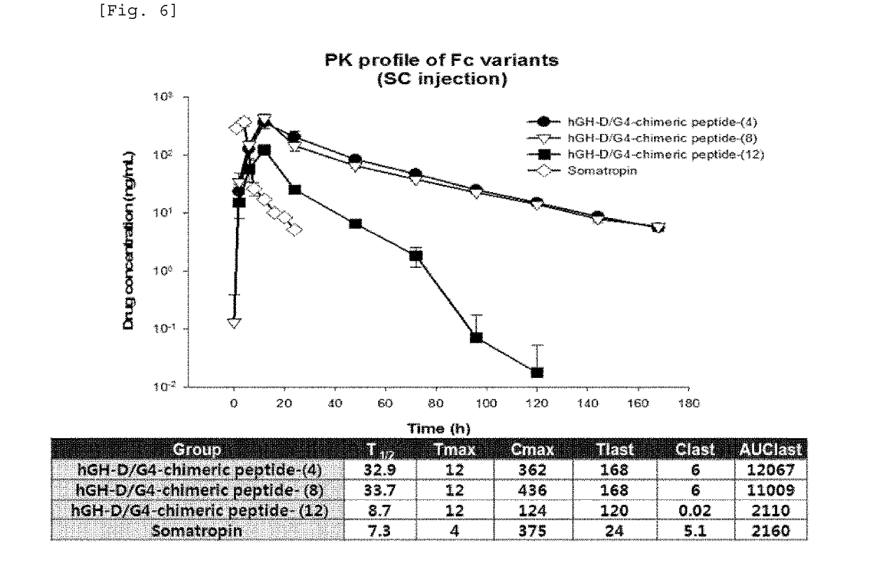

As shown in FIG. 6, hGH-chimeric IgG4 prepared by replacement and insertion of 4, 8 or 12 amino acids of IgD CH2 domain at the N-terminus of IgG4 CH2 domain showed remarkably increased in-vivo half-life, compared to the control group somatropin. In particular, the variant prepared by replacement and insertion of 4 or 8 amino acids of IgD CH2 domain showed the longest in-vivo half-life. Taken together, although variants are prepared by replacement with amino acid residues of IgD CH2 domain, their long half-life can be maintained.

### Example 5

### Preparation of Various CH2 Domain Variants

As confirmed in Examples 3 and 4, ADCC-inducing ability can be inhibited and in-vivo half-life of the fusion protein can be increased by switching of the N-terminus of CH2 domain of IgG4 with CH2 domain of different class of IgD having no FcγR binding site. Therefore, the biologically active protein can be safely used while effectively increasing its in-vivo half-life.

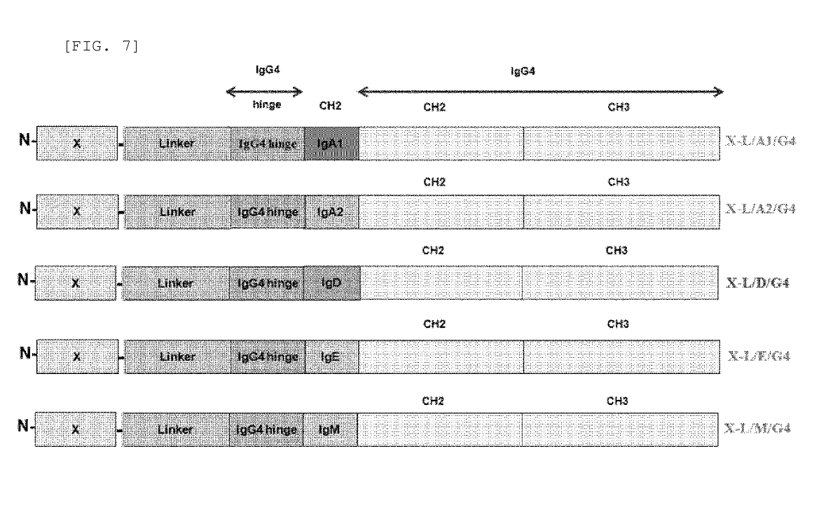

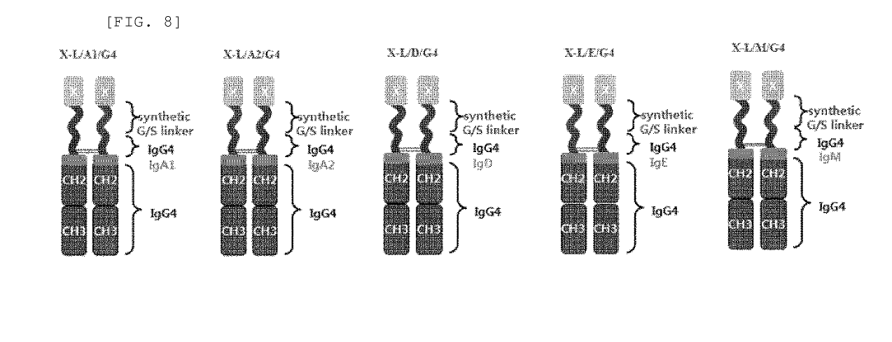

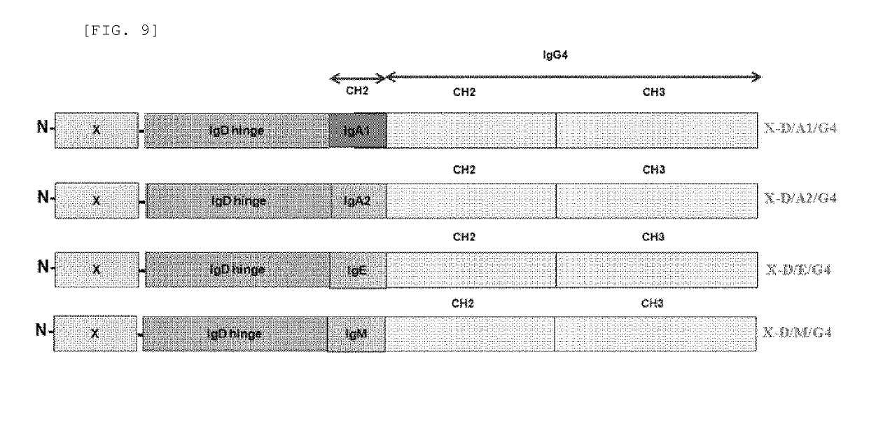

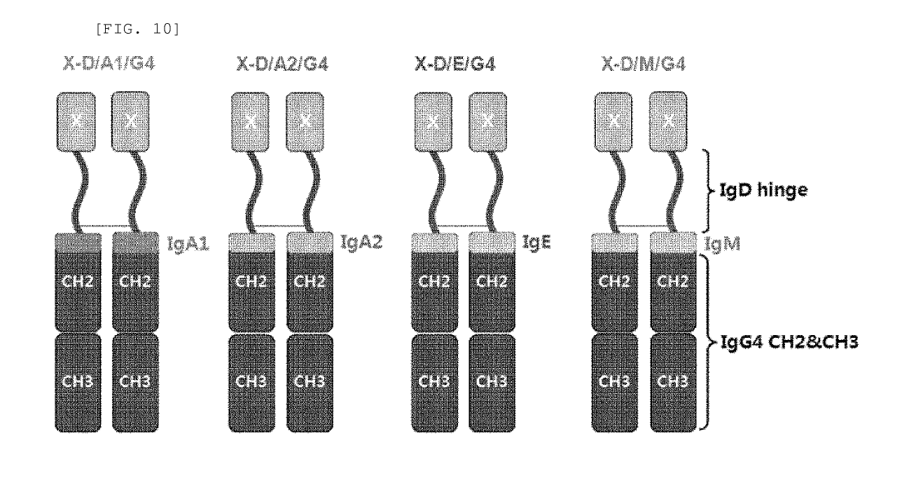

Accordingly, it was examined whether the same effects can be obtained by replacing the N-terminus of IgG4 CH2 domain with CH2 domain of different immunoglobulin having no FcγR binding site other than IgD. To achieve this, various IgG4 Fc CH2 variants (X-L/A1/G4, X-L/A2/G4, X-L/D/G4, X-L/E/G4 and X-L/M/G4) were prepared using CH2 domain sequences of IgA1, IgA2, IgE and IgM selected in Example 3, as shown in FIGS. 7 and 8. Additionally, as shown in FIGS. 9 and 10, the hinge region was replaced with IgD hinge so as to prepare various IgG4 Fc CH2 variants linked thereto (X-D/A1/G4, X-D/A2/G4, X-D/E/G4 and X-D/M/G4). In this regard, many variants were prepared by replacement of 4 to 18 amino acid residues as well as 8 amino acid residues of CH2 domain of each immunoglobulin.

Meanwhile, X represents the biologically active protein, and hGH (SEQ ID NO: 8) was applied thereto in the following Examples, as in the above Examples.

### Example 6

### Test of In-Vivo Half-Life of IgG4 Fc CH2 Variant-Fused Protein

In order to investigate the effects of the various IgG4 Fc CH2 variants prepared in Example 5 on in-vivo half-life of the biologically active protein, X-L/A1/G4, X-L/A2/G4, X-L/E/G4 and X-L/M/G4 (hGH applied to X) illustrated in FIGS. 7 and 8 were subjected to the experiment in the same manner as in Example 1.

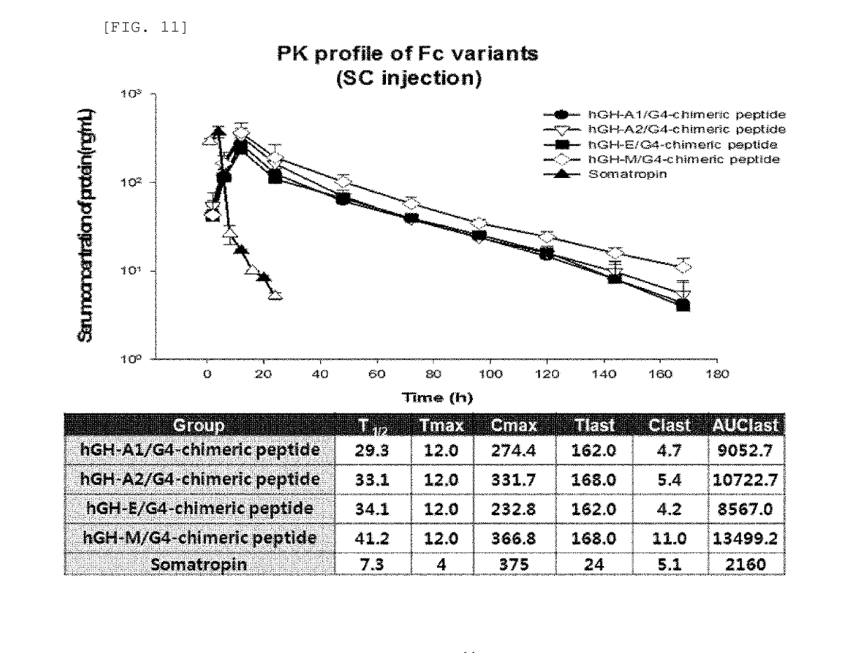

As shown in FIG. 11, it was found that each of the IgG4 Fc CH2 variants prepared in the present invention remarkably increased in-vivo half-life of the biologically active protein hGH, compared to the control group somatropin, indicating that the increased in-vivo half-life can be maintained by replacement with IgA1, IgA2, IgE and IgM CH2 domain as well as replacement with IgD CH2 domain as confirmed in Example 4.

### Example 7

### FcγRI-Binding Ability Assay of IgG4 Fc CH2 Variant-Fused Protein

In Example 6, it was confirmed that replacement of a portion of the N-terminus of IgG4 Fc CH2 domain with a portion of IgA1, IgA2, IgE or IgM CH2 domain increased in-vivo half-life, and the effect on ADCC-inducing ability by FcγRI binding was also examined.

X-L/A1/G4, X-L/A2/G4, X-L/E/G4 and X-L/M/G4 (hGH applied to X) prepared in Example 5, as illustrated in FIGS. 7 and 8, were subjected to the experiment in the same manner as in Example 2 in order to examine whether they bind to FcγRI.

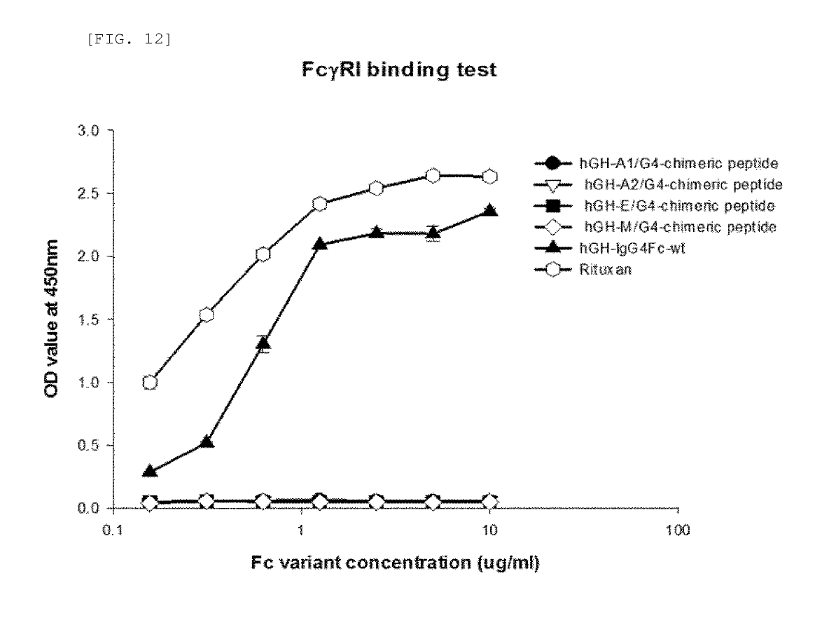

As shown in FIG. 12, the control rituxan and the wild-type IgG4 Fc-fused hGH showed a strong binding to FcγRI, whereas hGH fused with each IgG4 Fc CH2 variant prepared in the present invention showed no FcγRI-binding ability so as to induce no ADCC.

As confirmed in Examples 6 and 7, the IgG4 Fc CH2 variants prepared in the present invention maintain the long half-life and have no disadvantage of ADCC induction through FcγR binding to minimize undesirable immunogenicity in vivo when the wild-type IgG4 is used as a partner of a fusion protein. Therefore, the variants can be usefully applied to a variety of therapeutic proteins such as biologically active peptides or polypeptides having short in-vivo half-life.

### Example 8

### Hydrophobicity Profile of IgG4 Fc CH2 Variant

In Examples 6 and 7, the experiments were carried out using the variants prepared by replacement of 10 amino acid residues of the N-terminus of CH2 domain of the wild-type IgG4 Fc with 8 amino acid residues of IgA1, IgA2, IgE or IgM CH2 domain. It was intended to examine whether replacement of additional amino acid residues shows the same effect as the replacement of 8 amino acid residues and hydrophobicity was examined in order to determine the maximum replacement range.

In general, when a chimeric form is formed by switching of a portion of Ig domain with a portion of Ig domain of different class, the junction site shows immunogenicity to induce undesirable immune responses. Therefore, in the preparation of chimeric proteins, it is very important to maintain hydrophobicity in order to prevent exposure of the junction site to the exterior.

Therefore, the present inventors examined hydrophobicity profiles of the junction sites of IgG4 Fc CH2 variants prepared as above using an ExPASy—ProtScale program.

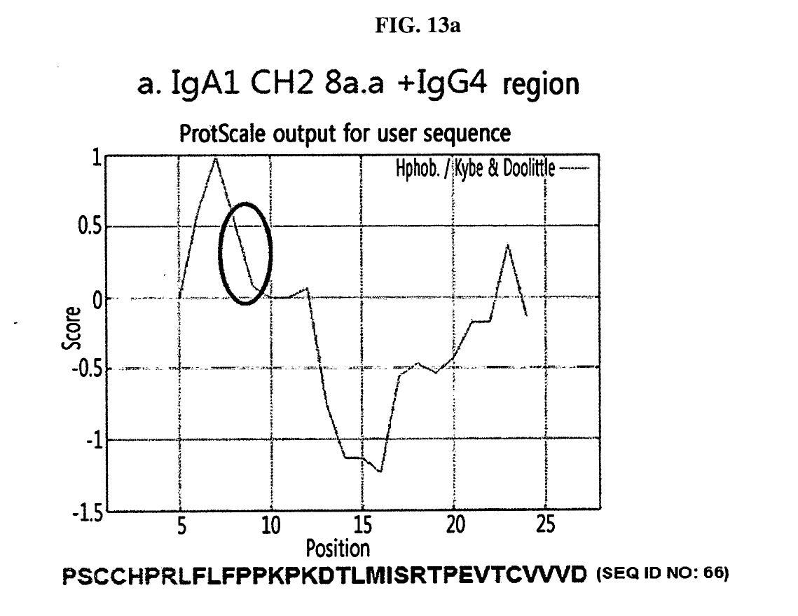

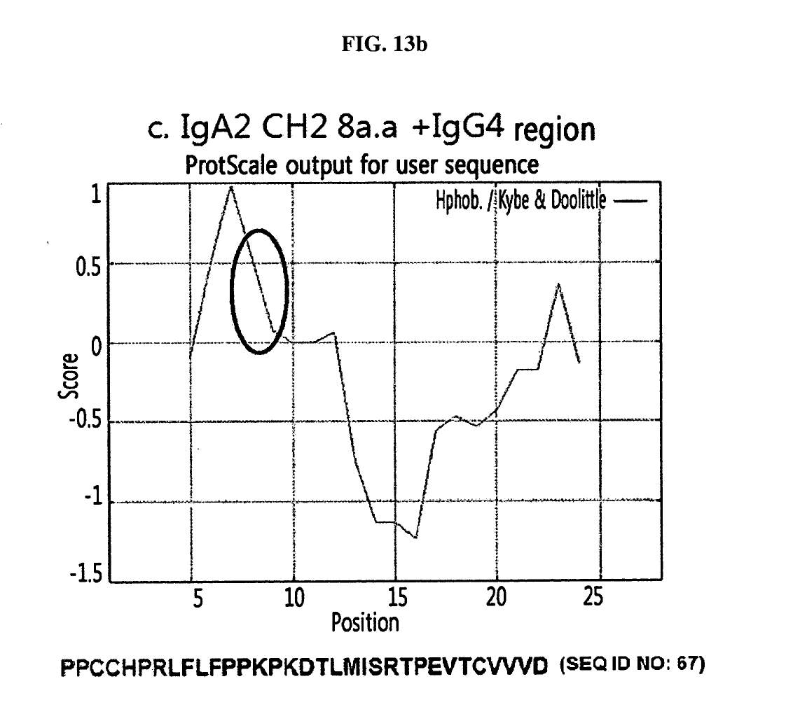

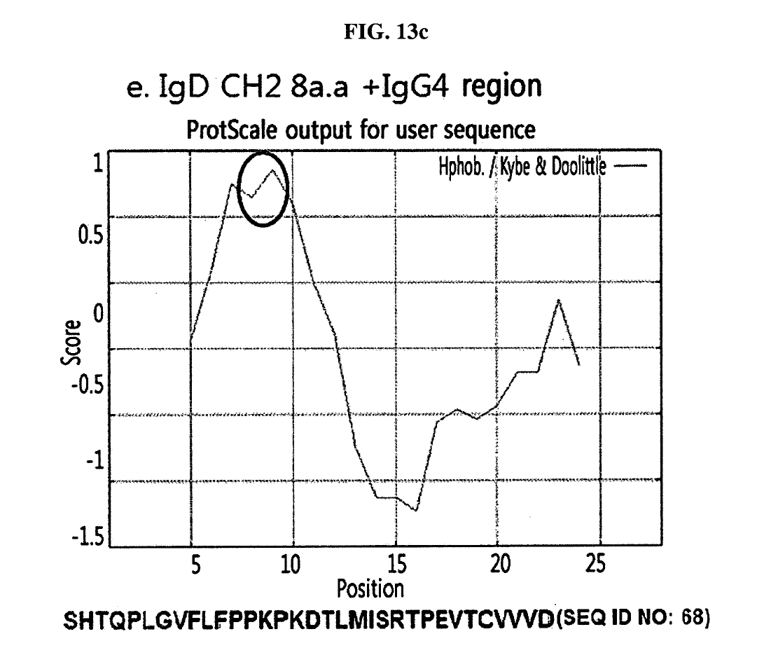

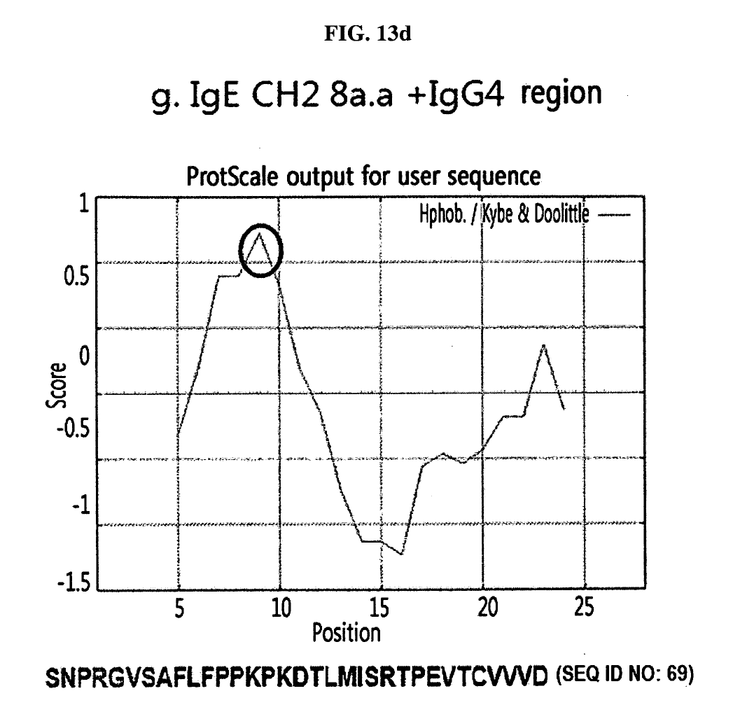

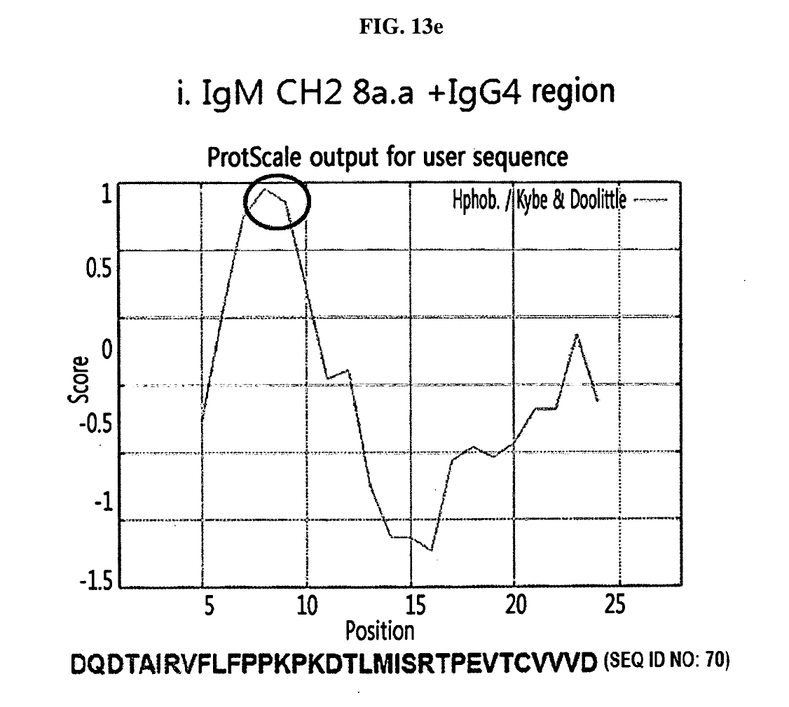

First, the variant prepared by replacement of a portion of the N-terminus of IgG4 Fc CH2 domain with 8 amino acid residues of IgA1, IgA2, IgD, IgE or IgM CH2 domain was examined. As shown in FIGS. 13a to 13e, the junction site at positions 8 to 9 has the positive value, indicating that it has hydrophobicity and thus no undesirable immune responses occur.

Next, replacements of various numbers of amino acid residues were carried out in addition to replacement of 8 amino acid residues. As a result, when 4 or more of amino acid residues were replaced, the junction sites also showed hydrophobicity.

Meanwhile, in order to determine the suitable replacement range, a larger number of amino acid residues were replaced and then the experiment was carried out. In detail, after 20 amino acid residues of the N-terminus of IgG4 Fc CH2 domain were replaced with 18 amino acid residues of IgA1, IgA2, IgD, IgE or IgM CH2 domain and 21 amino acid residues of the N-terminus of IgG4 Fc CH2 domain were replaced with 19 amino acid residues of IgA1, IgA2, IgD, IgE or IgM CH2 domain, the experiments were carried out. As shown in FIGS. 14a, 14b, 15a, 15b, 16a, 16b, 17a, 17b, 18a, and 18b, when they were replaced with 18 amino acid residues, the junction site at positions 18 to 19 had a high positive value, indicating hydrophobicity. In contrast, when they were replaced with 19 amino acid residues, the junction site at positions 19 to 20 had a relatively low value, indicating poor hydrophobicity profile. That is, if 18 amino acid residues are inserted, the junction site exists in the inner space to induce no undesirable immune responses. However, if 19 amino acid residues are inserted, the junction site is exposed to the exterior upon formation of a three-dimensional structure to increase immunogenicity.

### EFFECT OF THE INVENTION

When the CH2 polypeptide variant of IgG4 Fc of the present invention is used, half-life of a biologically active protein can be increased without inducing ADCC. Therefore, it can be usefully applied to different types of biologically active protein drugs having a short in-vivo half-life. Moreover, the junction site of the region to be replaced can be prepared to have hydrophobicity without artificial mutations in immunoglobulins, thereby minimizing non-specific immune responses.

What is claimed is:

1. A modified human IgG4 Fc polypeptide comprising a modified CH2 domain of human IgG4 Fc, and a CH3 domain of human IgG4 Fc in an N-terminal to C-terminal direction,
wherein the modification to the CH2 domain of human IgG4 Fc comprises a replacement of a 6-20 consecutive amino acid sequence from the N-terminus of the CH2 domain of IgG4 with one selected from the group consisting of a 4-18 consecutive amino acid sequence from the N-terminus of a CH2 domain of human IgA1 Fc, a 4-18 consecutive amino acid sequence from the N-terminus of a CH2 domain of human IgA2 Fc, a 4-18 consecutive amino acid sequence from the N-terminus of a CH2 domain of human IgD Fc, a 4-18 consecutive amino acid sequence from the N-terminus of a CH2 domain of human IgE Fc, and a 4-18 consecutive amino acid sequence from the N-terminus of a CH2 domain of human IgM Fc.

2. The modified polypeptide of claim 1, which consists of the following formula (I):
N′-(L)ₙ-(Z1)ₘ-(Y)ₒ—Z2-Z3-Z4-C′ (I)
wherein
N′ is the N-terminal of the polypeptide,
C′ is the C-terminal of the polypeptide,
Z1 is a CH1 domain of IgG4 (SEQ ID NO: 54),
L is a linker;
Y is a hinge of IgG4 or IgD; Z2-Z3 is the modified CH2 domain of IgG4, wherein Z2 is one selected from the group consisting of a 4-18 consecutive amino acid sequence starting from position 1 of SEQ ID NO: 47, a 4-18 consecutive amino acid sequence starting from position 1 of SEQ ID NO: 48, a 4-18 consecutive amino acid sequence starting from position 1 of SEQ ID NO: 49, a 4-18 consecutive amino acid sequence starting from position 1 of SEQ ID NO: 50, and a 4-18 consecutive amino acid sequence starting from position 1 of SEQ ID NO: 51; and Z3 is a 90-104 consecutive amino acid sequence starting from the C-terminus of SEQ ID NO: 52;
Z4 is the CH3 domain of IgG4 (SEQ ID NO: 53);
m is an integer of 0 or 1;
n is an integer of 0 or 1; and
o is an integer or 0 or 1.

3. The modified polypeptide of claim 2, wherein Z2 is one selected from the group consisting of a 4-12 consecutive amino acid sequence starting from position 1 of SEQ ID NO: 47, a 4-12 consecutive amino acid sequence starting from position 1 of SEQ ID NO: 48, a 4-12 consecutive amino acid sequence starting from position 1 of SEQ ID NO: 49, a 4-12 consecutive amino acid sequence starting from position 1 of SEQ ID NO: 50, and a 4-12 consecutive amino acid sequence starting from position 1 of SEQ ID NO: 51; and Z3 is a 90-104 consecutive amino acid sequence starting from the C-terminus of SEQ ID NO: 52.

4. The modified polypeptide of claim 2, wherein m is 0 and n is 1.

5. The modified polypeptide of claim 2, wherein Z2 is one selected from the group consisting of a 8 consecutive amino acid sequence starting from position 1 of SEQ ID NO: 47, a 8 consecutive amino acid sequence starting from position 1 of SEQ ID NO: 48, a 8 consecutive amino acid sequence starting from position 1 of SEQ ID NO: 49, a 8 consecutive amino acid sequence starting from position 1 of SEQ ID NO: 50, and a 8 consecutive amino acid sequence starting from position 1 of SEQ ID NO: 51.

6. The modified polypeptide of claim 2, wherein Y is a 5-12 consecutive amino acid sequence starting from the C-terminus of SEQ ID NO: 45 or a 5-64 consecutive amino acid sequence starting from the C-terminus of SEQ ID NO: 46.

7. The modified polypeptide of claim 2, wherein the linker is a GS oligopeptide linker.

8. The modified polypeptide according to claim 1, wherein the polypeptide comprises an amino acid sequence selected from the group consisting of SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

9. A chimeric polypeptide comprising the modified polypeptide of claim 1, and a biologically active molecule, said polypeptide being coupled to the biologically active molecule, wherein the chimeric polypeptide shows an increased circulating half-life than that of the native form of the biologically active molecule.

10. The chimeric polypeptide according to claim 9, wherein the biologically active molecule is a hormone, a cytokine, a growth factor, a co-stimulatory molecule, a hormone receptor, a cytokine receptor, a growth factor receptor, or a short peptide.

11. The chimeric polypeptide according to claim 9, wherein the polypeptide and the biologically active molecule are coupled to each other via a linker.

12. The chimeric polypeptide according to claim 10,
wherein the linker is a peptide of 10 to 20 amino acid residues consisting of Gly and Ser residues.

13. The chimeric polypeptide according to claim 9, wherein the chimeric polypeptide comprises an amino acid sequence selected from the group consisting of SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, and SEQ ID NO: 26.

14. A method for producing the polypeptide of claim 1, comprising (i) introducing a nucleic acid molecule encoding the polypeptide of claim 1 into a mammalian host cell; (ii) culturing the cell under conditions where the polypeptide can be expressed; and (iii) harvesting the expressed polypeptide.

15. An isolated nucleic acid molecule encoding the modified polypeptide of claim 1.

16. The nucleic acid molecule according to claim 15, wherein the nucleic acid molecule comprises a nucleotide sequence selected from the group consisting of SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34 and SEQ ID NO: 35.

17. The nucleic acid molecule according to claim 15, wherein the nucleic acid molecule further comprises a signal sequence or a leader sequence.

18. The nucleic acid molecule according to claim 17, wherein the signal sequence is a tPa signal sequence.

19. An isolated nucleic acid molecule encoding the chimeric polypeptide of claim 9.

20. The nucleic acid molecule according to claim 19, wherein the nucleic acid molecule comprises a nucleotide sequence selected from the group consisting of SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, and SEQ ID NO: 44.

21. The nucleic acid molecule according to claim 19, wherein the nucleic acid molecule further comprises a signal sequence or a leader sequence.

22. The nucleic acid molecule according to claim 21, wherein the signal sequence is a tPa signal sequence.

23. An expression vector comprising the nucleic acid molecule of claim 15.

24. An expression vector comprising the nucleic acid molecule of claim 19.

25. A host cell comprising the expression vector of claim 23.

26. A host cell comprising the expression vector of claim 24.

27. A method for producing the polypeptide of claim 9, comprising (i) introducing a nucleic acid molecule encoding the polypeptide of claim 9 into a mammalian host cell; (ii) culturing the cell under conditions where the polypeptide can be expressed; and (iii) harvesting the expressed polypeptide.


SEQUENCE LISTING (80 sequences)

SEQ ID NO: 1 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPDRDLCGCYSVSSVLSGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 3 - protein, 340 aa, Homo sapiens
ASPTSPKVFPLSLDSTPQDGNVVVACLVQGFFPQZPLSVTWSESGZNVTARNFPPSQBASGDLYTTSSQLTLPATZCPDGKSVTCHVKHYTNSSQDVTVPCRVPPPPPCCHPRLSLHRPALEDLLLGSEANLTCTLTGLRDASGATFTWTPSSGKSAVEGPPERDLCGCYSVSSVLPGCAZPWNHGETFTCTAAHPELKTPLTANITKSGBTFRPEVHLLPPPSEELALBZLVTLTCLARGFSPKBVLVRWLQGSQELPREKYLTWASRQZPSZGTTTYAVTSILRVAAZBWKKGETFSCMVGHEALPLAFTQKTIDRLAGKPTHINVSVVMAEABGTCY

SEQ ID NO: 4 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 5 - protein, 424 aa, Homo sapiens
SPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 6 - protein, 449 aa, Homo sapiens
APTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 7 - protein, 15 aa, Artificial Sequence
  GS linker
GGGGSGGGGSGGGGS

SEQ ID NO: 8 - protein, 217 aa, Homo sapiens
MATGSRTSLLLAFGLLCLPWLQEGSAFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 9 - protein, 227 aa, Artificial Sequence
  Sequence of X-L/A1/G4 Fc domain
ESKYGPPCPSCPPSCCHPRLFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 227 aa, Artificial Sequence
  Sequence of X-L/A2/G4 Fc domain
ESKYGPPCPSCPPPCCHPRLFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 227 aa, Artificial Sequence
  Sequence of X-L/D/G4 Fc domain
ESKYGPPCPSCPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 227 aa, Artificial Sequence
  Sequence of X-L/E/G4 Fc domain
ESKYGPPCPSCPSNPRGVSAFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 227 aa, Artificial Sequence
  Sequence of X-L/M/G4 Fc domain
ESKYGPPCPSCPDQDTAIRVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 245 aa, Artificial Sequence
  Sequence of X-D/A1/G4 Fc domain
RNTGRGGEEKKKEKEKEEQEERETKTPECPPSCCHPRLFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 245 aa, Artificial Sequence
  Sequence of X-D/A2/G4 Fc domain
RNTGRGGEEKKKEKEKEEQEERETKTPECPPPCCHPRLFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 245 aa, Artificial Sequence
  Sequence of X-D/E/G4 Fc domain
RNTGRGGEEKKKEKEKEEQEERETKTPECPSNPRGVSAFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 245 aa, Artificial Sequence
  Sequence of X-D/M/G4 Fc domain
RNTGRGGEEKKKEKEKEEQEERETKTPECPDQDTAIRVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 432 aa, Artificial Sequence
  Sequence of hGH-L/A1/G4
FPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGGGSGGGGSGGGGESKYGPPCPSCPPSCCHPRLFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 432 aa, Artificial Sequence
  Sequence of hGH-L/A2/G4
FPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGGGSGGGGSGGGGESKYGPPCPSCPPPCCHPRLFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 432 aa, Artificial Sequence
  Sequence of hGH-L/D/G4
FPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGGGSGGGGSGGGGESKYGPPCPSCPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 432 aa, Artificial Sequence
  Sequence of hGH-L/E/G4
FPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGGGSGGGGSGGGGESKYGPPCPSCPSNPRGVSAFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 432 aa, Artificial Sequence
  Sequence of hGH-L/M/G4
FPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGGGSGGGGSGGGGESKYGPPCPSCPDQDTAIRVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 436 aa, Artificial Sequence
  Sequence of hGH-D/A1/G4
FPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFRNTGRGGEEKKKEKEKEEQEERETKTPECPPSCCHPRLFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 436 aa, Artificial Sequence
  Sequence of hGH-D/A2/G4
FPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFRNTGRGGEEKKKEKEKEEQEERETKTPECPPPCCHPRLFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 436 aa, Artificial Sequence
  Sequence of hGH-D/E/G4
FPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFRNTGRGGEEKKKEKEKEEQEERETKTPECPSNPRGVSAFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 436 aa, Artificial Sequence
  Sequence of hGH-D/M/G4
FPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFRNTGRGGEEKKKEKEKEEQEERETKTPECPDQDTAIRVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - DNA, 684 bp, Artificial Sequence
  Sequence of X-L/A1/G4 Fc domain
gagagcaagtacggacctccctgccccagctgtcccccttcttgctgtcaccccagactgttcctgtttccccccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtga

SEQ ID NO: 28 - DNA, 684 bp, Artificial Sequence
  Sequence of X-L/A2/G4 Fc domain
gagagcaagtacggacctccctgccccagctgtccccctccctgctgtcaccccagactgttcctgtttccccccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtga

SEQ ID NO: 29 - DNA, 684 bp, Artificial Sequence
  Sequence of X-L/D/G4 Fc domain
gagagcaagtacggacctccctgccccagctgtcccagccacacccagcccctgggcgtgttcctgtttccccccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtga

SEQ ID NO: 30 - DNA, 684 bp, Artificial Sequence
  Sequence of X-L/E/G4 Fc domain
gagagcaagtacggacctccctgccccagctgtcccagcaaccccagaggcgtgagcgccttcctgtttccccccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtga

SEQ ID NO: 31 - DNA, 684 bp, Artificial Sequence
  Sequence of X-L/M/G4 Fc domain
gagagcaagtacggacctccctgccccagctgtcccgaccaggacaccgccatcagagtgttcctgtttccccccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtga

SEQ ID NO: 32 - DNA, 738 bp, Artificial Sequence
  Sequence of X-D/A1/G4 Fc domain
cgcaacaccggccgcggcggcgaggagaagaagaaggagaaggagaaggaggagcaggaggagcgcgagaccaagacccccgagtgccccccttcttgctgtcaccccagactgttcctgtttccccccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtga

SEQ ID NO: 33 - DNA, 738 bp, Artificial Sequence
  Sequence of X-D/A2/G4 Fc domain
cgcaacaccggccgcggcggcgaggagaagaagaaggagaaggagaaggaggagcaggaggagcgcgagaccaagacccccgagtgcccccctccctgctgtcaccccagactgttcctgtttccccccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtga

SEQ ID NO: 34 - DNA, 738 bp, Artificial Sequence
  Sequence of X-D/E/G4 Fc domain
cgcaacaccggccgcggcggcgaggagaagaagaaggagaaggagaaggaggagcaggaggagcgcgagaccaagacccccgagtgccccagcaaccccagaggcgtgagcgccttcctgtttccccccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtga

SEQ ID NO: 35 - DNA, 738 bp, Artificial Sequence
  X-D/M/G4 Fc domain
cgcaacaccggccgcggcggcgaggagaagaagaaggagaaggagaaggaggagcaggaggagcgcgagaccaagacccccgagtgccccgaccaggacaccgccatcagagtgttcctgtttccccccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtga

SEQ ID NO: 36 - DNA, 1380 bp, Artificial Sequence
  Sequence of hGH-L/A1/G4
atggccaccggcagcagaaccagcctgctgctggccttcggcctgctgtgcctgccctggctccaggagggcagcgccttccccaccatccccctgagcagactgttcgacaacgccatgctgagagcccacagactgcaccagctggccttcgacacctaccaggagttcgaggaggcctacatccccaaggagcagaagtacagcttcctccagaacccccagaccagcctgtgcttcagcgagagcatccccacccccagcaacagagaggagacccagcagaagagcaacctggagctgctgagaatcagcctgctcctgatccagagctggctggagcccgtgcagttcctgagaagcgtgttcgccaacagcctggtgtacggcgccagcgacagcaacgtgtacgacctgctgaaggacctggaggagggcatccagaccctgatgggcagactggaggacggcagccccagaaccggccagatcttcaagcagacctacagcaagttcgacaccaacagccacaacgacgacgccctgctgaagaactacggcctgctgtactgcttcagaaaggacatggacaaggtggagaccttcctgagaatcgtgcagtgcagaagcgtggagggcagctgcggcttcggcggaggaggcagtggtggaggcggctctggcggtgggggatctgagagcaagtacggacctccctgccccagctgtcccccttcttgctgtcaccccagactgttcctgtttccccccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtga

SEQ ID NO: 37 - DNA, 1380 bp, Artificial Sequence
  Sequence of hGH-L/A2/G4
atggccaccggcagcagaaccagcctgctgctggccttcggcctgctgtgcctgccctggctccaggagggcagcgccttccccaccatccccctgagcagactgttcgacaacgccatgctgagagcccacagactgcaccagctggccttcgacacctaccaggagttcgaggaggcctacatccccaaggagcagaagtacagcttcctccagaacccccagaccagcctgtgcttcagcgagagcatccccacccccagcaacagagaggagacccagcagaagagcaacctggagctgctgagaatcagcctgctcctgatccagagctggctggagcccgtgcagttcctgagaagcgtgttcgccaacagcctggtgtacggcgccagcgacagcaacgtgtacgacctgctgaaggacctggaggagggcatccagaccctgatgggcagactggaggacggcagccccagaaccggccagatcttcaagcagacctacagcaagttcgacaccaacagccacaacgacgacgccctgctgaagaactacggcctgctgtactgcttcagaaaggacatggacaaggtggagaccttcctgagaatcgtgcagtgcagaagcgtggagggcagctgcggcttcggcggaggaggcagtggtggaggcggctctggcggtgggggatctgagagcaagtacggacctccctgccccagctgtccccctccctgctgtcaccccagactgttcctgtttccccccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtga

SEQ ID NO: 38 - DNA, 1380 bp, Artificial Sequence
  Sequence of hGH-L/D/G4
atggccaccggcagcagaaccagcctgctgctggccttcggcctgctgtgcctgccctggctccaggagggcagcgccttccccaccatccccctgagcagactgttcgacaacgccatgctgagagcccacagactgcaccagctggccttcgacacctaccaggagttcgaggaggcctacatccccaaggagcagaagtacagcttcctccagaacccccagaccagcctgtgcttcagcgagagcatccccacccccagcaacagagaggagacccagcagaagagcaacctggagctgctgagaatcagcctgctcctgatccagagctggctggagcccgtgcagttcctgagaagcgtgttcgccaacagcctggtgtacggcgccagcgacagcaacgtgtacgacctgctgaaggacctggaggagggcatccagaccctgatgggcagactggaggacggcagccccagaaccggccagatcttcaagcagacctacagcaagttcgacaccaacagccacaacgacgacgccctgctgaagaactacggcctgctgtactgcttcagaaaggacatggacaaggtggagaccttcctgagaatcgtgcagtgcagaagcgtggagggcagctgcggcttcggcggaggaggcagtggtggaggcggctctggcggtgggggatctgagagcaagtacggacctccctgccccagctgtcccagccacacccagcccctgggcgtgttcctgtttccccccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtga

SEQ ID NO: 39 - DNA, 1380 bp, Artificial Sequence
  Sequence of hGH-L/E/G4
atggccaccggcagcagaaccagcctgctgctggccttcggcctgctgtgcctgccctggctccaggagggcagcgccttccccaccatccccctgagcagactgttcgacaacgccatgctgagagcccacagactgcaccagctggccttcgacacctaccaggagttcgaggaggcctacatccccaaggagcagaagtacagcttcctccagaacccccagaccagcctgtgcttcagcgagagcatccccacccccagcaacagagaggagacccagcagaagagcaacctggagctgctgagaatcagcctgctcctgatccagagctggctggagcccgtgcagttcctgagaagcgtgttcgccaacagcctggtgtacggcgccagcgacagcaacgtgtacgacctgctgaaggacctggaggagggcatccagaccctgatgggcagactggaggacggcagccccagaaccggccagatcttcaagcagacctacagcaagttcgacaccaacagccacaacgacgacgccctgctgaagaactacggcctgctgtactgcttcagaaaggacatggacaaggtggagaccttcctgagaatcgtgcagtgcagaagcgtggagggcagctgcggcttcggcggaggaggcagtggtggaggcggctctggcggtgggggatctgagagcaagtacggacctccctgccccagctgtcccagcaaccccagaggcgtgagcgccttcctgtttccccccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtga

SEQ ID NO: 40 - DNA, 1380 bp, Artificial Sequence
  Sequence of hGH-L/M/G4
atggccaccggcagcagaaccagcctgctgctggccttcggcctgctgtgcctgccctggctccaggagggcagcgccttccccaccatccccctgagcagactgttcgacaacgccatgctgagagcccacagactgcaccagctggccttcgacacctaccaggagttcgaggaggcctacatccccaaggagcagaagtacagcttcctccagaacccccagaccagcctgtgcttcagcgagagcatccccacccccagcaacagagaggagacccagcagaagagcaacctggagctgctgagaatcagcctgctcctgatccagagctggctggagcccgtgcagttcctgagaagcgtgttcgccaacagcctggtgtacggcgccagcgacagcaacgtgtacgacctgctgaaggacctggaggagggcatccagaccctgatgggcagactggaggacggcagccccagaaccggccagatcttcaagcagacctacagcaagttcgacaccaacagccacaacgacgacgccctgctgaagaactacggcctgctgtactgcttcagaaaggacatggacaaggtggagaccttcctgagaatcgtgcagtgcagaagcgtggagggcagctgcggcttcggcggaggaggcagtggtggaggcggctctggcggtgggggatctgagagcaagtacggacctccctgccccagctgtcccgaccaggacaccgccatcagagtgttcctgtttccccccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtga

SEQ ID NO: 41 - DNA, 1389 bp, Artificial Sequence
  Sequence of hGH-D/A1/G4
atggccaccggcagcagaaccagcctgctgctggccttcggcctgctgtgcctgccctggctccaggagggcagcgccttccccaccatccccctgagcagactgttcgacaacgccatgctgagagcccacagactgcaccagctggccttcgacacctaccaggagttcgaggaggcctacatccccaaggagcagaagtacagcttcctccagaacccccagaccagcctgtgcttcagcgagagcatccccacccccagcaacagagaggagacccagcagaagagcaacctggagctgctgagaatcagcctgctcctgatccagagctggctggagcccgtgcagttcctgagaagcgtgttcgccaacagcctggtgtacggcgccagcgacagcaacgtgtacgacctgctgaaggacctggaggagggcatccagaccctgatgggcagactggaggacggcagccccagaaccggccagatcttcaagcagacctacagcaagttcgacaccaacagccacaacgacgacgccctgctgaagaactacggcctgctgtactgcttcagaaaggacatggacaaggtggagaccttcctgagaatcgtgcagtgcagaagcgtggagggcagctgcggcttccgcaacaccggccgcggcggcgaggagaagaagaaggagaaggagaaggaggagcaggaggagcgcgagaccaagacccccgagtgccccccttcttgctgtcaccccagactgttcctgtttccccccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtga

SEQ ID NO: 42 - DNA, 1389 bp, Artificial Sequence
  Sequence of hGH-D/A2/G4
atggccaccggcagcagaaccagcctgctgctggccttcggcctgctgtgcctgccctggctccaggagggcagcgccttccccaccatccccctgagcagactgttcgacaacgccatgctgagagcccacagactgcaccagctggccttcgacacctaccaggagttcgaggaggcctacatccccaaggagcagaagtacagcttcctccagaacccccagaccagcctgtgcttcagcgagagcatccccacccccagcaacagagaggagacccagcagaagagcaacctggagctgctgagaatcagcctgctcctgatccagagctggctggagcccgtgcagttcctgagaagcgtgttcgccaacagcctggtgtacggcgccagcgacagcaacgtgtacgacctgctgaaggacctggaggagggcatccagaccctgatgggcagactggaggacggcagccccagaaccggccagatcttcaagcagacctacagcaagttcgacaccaacagccacaacgacgacgccctgctgaagaactacggcctgctgtactgcttcagaaaggacatggacaaggtggagaccttcctgagaatcgtgcagtgcagaagcgtggagggcagctgcggcttccgcaacaccggccgcggcggcgaggagaagaagaaggagaaggagaaggaggagcaggaggagcgcgagaccaagacccccgagtgcccccctccctgctgtcaccccagactgttcctgtttccccccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtga

SEQ ID NO: 43 - DNA, 1389 bp, Artificial Sequence
  Sequence of hGH-D/E/G4
atggccaccggcagcagaaccagcctgctgctggccttcggcctgctgtgcctgccctggctccaggagggcagcgccttccccaccatccccctgagcagactgttcgacaacgccatgctgagagcccacagactgcaccagctggccttcgacacctaccaggagttcgaggaggcctacatccccaaggagcagaagtacagcttcctccagaacccccagaccagcctgtgcttcagcgagagcatccccacccccagcaacagagaggagacccagcagaagagcaacctggagctgctgagaatcagcctgctcctgatccagagctggctggagcccgtgcagttcctgagaagcgtgttcgccaacagcctggtgtacggcgccagcgacagcaacgtgtacgacctgctgaaggacctggaggagggcatccagaccctgatgggcagactggaggacggcagccccagaaccggccagatcttcaagcagacctacagcaagttcgacaccaacagccacaacgacgacgccctgctgaagaactacggcctgctgtactgcttcagaaaggacatggacaaggtggagaccttcctgagaatcgtgcagtgcagaagcgtggagggcagctgcggcttccgcaacaccggccgcggcggcgaggagaagaagaaggagaaggagaaggaggagcaggaggagcgcgagaccaagacccccgagtgccccagcaaccccagaggcgtgagcgccttcctgtttccccccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtga

SEQ ID NO: 44 - DNA, 1389 bp, Artificial Sequence
  Sequence of hGH-D/M/G4
atggccaccggcagcagaaccagcctgctgctggccttcggcctgctgtgcctgccctggctccaggagggcagcgccttccccaccatccccctgagcagactgttcgacaacgccatgctgagagcccacagactgcaccagctggccttcgacacctaccaggagttcgaggaggcctacatccccaaggagcagaagtacagcttcctccagaacccccagaccagcctgtgcttcagcgagagcatccccacccccagcaacagagaggagacccagcagaagagcaacctggagctgctgagaatcagcctgctcctgatccagagctggctggagcccgtgcagttcctgagaagcgtgttcgccaacagcctggtgtacggcgccagcgacagcaacgtgtacgacctgctgaaggacctggaggagggcatccagaccctgatgggcagactggaggacggcagccccagaaccggccagatcttcaagcagacctacagcaagttcgacaccaacagccacaacgacgacgccctgctgaagaactacggcctgctgtactgcttcagaaaggacatggacaaggtggagaccttcctgagaatcgtgcagtgcagaagcgtggagggcagctgcggcttccgcaacaccggccgcggcggcgaggagaagaagaaggagaaggagaaggaggagcaggaggagcgcgagaccaagacccccgagtgccccgaccaggacaccgccatcagagtgttcctgtttccccccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtga

SEQ ID NO: 45 - protein, 12 aa, Artificial Sequence
  Synthetic construct
ESKYGPPCPSCP

SEQ ID NO: 46 - protein, 64 aa, Artificial Sequence
  Synthetic construct
RWPESPKAQASSVPTAQPQAEGSLAKATTAPATTRNTGRGGEEKKKEKEKEEQEERETKTPECP

SEQ ID NO: 47 - protein, 18 aa, Artificial Sequence
  Synthetic construct
PSCCHPRLSLHRPALEDL

SEQ ID NO: 48 - protein, 32 aa, Artificial Sequence
  Synthetic construct
FPTIPLSRLFDNAMLRAHRLHQLAFDTYQEFE

SEQ ID NO: 49 - protein, 18 aa, Artificial Sequence
  Synthetic construct
PPCCHPRLSLHRPALEDL

SEQ ID NO: 50 - protein, 18 aa, Artificial Sequence
  Synthetic construct
SNPRGVSAYLSRPSPFDL

SEQ ID NO: 51 - protein, 18 aa, Artificial Sequence
  Synthetic construct
DQDTAIRVFAIPPSFASI

SEQ ID NO: 52 - protein, 104 aa, Artificial Sequence
  Synthetic construct
GPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 53 - protein, 107 aa, Artificial Sequence
  Synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 93 aa, Artificial Sequence
  Synthetic construct
PSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 55 - protein, 6 aa, Artificial Sequence
  Synthetic construct
FLGGPS

SEQ ID NO: 56 - protein, 56 aa, Homo sapiens
  IgG1 CH2 region
PELLGGPSVFLFPPKPKDTLMISRTPEVICVVVDVSHEDPEVKFNWYVDGVEVHNA

SEQ ID NO: 57 - protein, 56 aa, Homo sapiens
  IgG3 CH2 region
PELLGGPSVFLFPPKPKDTIMISRTPEVTCVVVDVSHEDPEVQFKWYVDGVEVHNA

SEQ ID NO: 58 - protein, 56 aa, Homo sapiens
  IgG4 CH2 region
PEFLGGPSVFLFPPKPKDTLMISRTPEVTCVVVDVSQEDPEVQFNWYVDGVEVHNA

SEQ ID NO: 59 - protein, 55 aa, Homo sapiens
  IgG1 CH2 region
PPVAGPSVFLFPPKPKDTLMISRTPEVTCVVVDVSHEDPEVQFNWYVDGVEVHNA

SEQ ID NO: 60 - protein, 58 aa, Homo sapiens
  IgE CH2 region
KCADSNPRGVSAYLSRPSPFDLFIRKSPTITCLVVDLAPSKGTVNLTWSRASGKFVNH

SEQ ID NO: 61 - protein, 54 aa, Homo sapiens
  IgA1 CH2 region
TPSPSCCHPRLSLHRPALEDLLLGSEANLTCTLTGLRDASGVTFTWTPSSGKSA

SEQ ID NO: 62 - protein, 54 aa, Homo sapiens
  IgA2 CH2 region
PPPPPCCHPRLSLHRPALEDLLLGSEANLTCTLTGLRDASGATFTWTPSSGKSA

SEQ ID NO: 63 - protein, 57 aa, Homo sapiens
  IgM CH2 region
MCVPDQDTAIRVFAIPPSFASIFLTKSTKLTCLVTDLTTYDSVTISWTRQNGEAVKT

SEQ ID NO: 64 - protein, 55 aa, Homo sapiens
  IgD CH2 region
CPSHTQPLGVYLLTPAVQDLWLRDKATFTCFVVGSDLKDAHLTWEVAGKVPTGGV

SEQ ID NO: 65 - protein, 25 aa, Aritifical sequence
  Synthetic construct
CPSCPAPEFLGGPSVFLFPPKPKDT

SEQ ID NO: 66 - protein, 33 aa, Aritifical sequence
  Synthetic construct
PSCCHPRLFLFPPKPKDTLMISRTPEVTCVVVD

SEQ ID NO: 67 - protein, 33 aa, Aritifical sequence
  Synthetic construct
PPCCHPRLFLFPPKPKDTLMISRTPEVTCVVVD

SEQ ID NO: 68 - protein, 33 aa, Aritifical sequence
  Synthetic construct
SHTQPLGVFLFPPKPKDTLMISRTPEVTCVVVD

SEQ ID NO: 69 - protein, 33 aa, Aritifical sequence
  Synthetic construct
SNPRGVSAFLFPPKPKDTLMISRTPEVTCVVVD

SEQ ID NO: 70 - protein, 33 aa, Aritifical sequence
  Synthetic construct
DQDTAIRVFLFPPKPKDTLMISRTPEVTCVVVD

SEQ ID NO: 71 - protein, 33 aa, Aritifical sequence
  Synthetic construct
PSCCHPRLSLHRPALEDLLMISRTPEVTCVVVD

SEQ ID NO: 72 - protein, 33 aa, Aritifical sequence; identical to SEQ ID NO: 71
  Synthetic construct

SEQ ID NO: 73 - protein, 33 aa, Aritifical sequence
  Synthetic construct
PPCCHPRLSLHRPALEDLLMISRTPEVTCVVVD

SEQ ID NO: 74 - protein, 33 aa, Aritifical sequence; identical to SEQ ID NO: 73
  Synthetic construct

SEQ ID NO: 75 - protein, 33 aa, Aritifical sequence
  Synthetic construct
SHTQPLGVYLLTPAVQDLLMISRTPEVTCVVVD

SEQ ID NO: 76 - protein, 33 aa, Aritifical sequence
  Synthetic construct
SHTQPLGVYLLTPAVQDLWMISRTPEVTCVVVD

SEQ ID NO: 77 - protein, 33 aa, Aritifical sequence
  Synthetic construct
SNPRGVSAYLSRPSPFDLLMISRTPEVTCVVVD

SEQ ID NO: 78 - protein, 33 aa, Aritifical sequence
  Synthetic construct
SNPRGVSAYLSRPSPFDLFMISRTPEVTCVVVD

SEQ ID NO: 79 - protein, 38 aa, Aritifical sequence
  Synthetic construct
DQDTAIRVFAIPPSFASILMISRTPEVTCVVVDVSQED

SEQ ID NO: 80 - protein, 33 aa, Aritifical sequence
  Synthetic construct
DQDTAIRVFAIPPSFASIFMISRTPEVTCVVVD